SEQUENCE LISTING (59 sequences)

SEQ ID NO: 1 - protein, 231 aa, Artificial Sequence
  source: /note=“Description of Artificial Sequence Synthetic polypeptide”
MSTESMIRDVELAEEALPKKTGGPQGSRRCLFLSLFSFLIVAGATTLFCLLHFGVIGPQREEFPRDLSLISPLAQARSSSRTPSDKVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 2 - DNA, 696 bp, Artificial Sequence
  source: /note=“Description of Artificial Sequence Synthetic polynucleotide”
atgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcaagatcatcttctcgaaccccgagtgacaaggtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtcg

SEQ ID NO: 3 - protein, 220 aa, Artificial Sequence
  source: /note=“Description of Artificial Sequence Synthetic polypeptide”
MSTESMIRDVELAEEALPKKTGGPQGSRRCLFLSLFSFLIVAGATTLFCLLHFGVIGPQREEFPRDLSLISPLAQAAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 4 - DNA, 663 bp, Artificial Sequence
  source: /note=“Description of Artificial Sequence Synthetic polynucleotide”
atgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtcg

SEQ ID NO: 5 - protein, 231 aa, Artificial Sequence
  source: /note=“Description of Artificial Sequence Synthetic polypeptide”
MSTESMIRDVELAEEALPKKTGGPQGSRRCLFLSLFSFLIVAGATTLFCLLHFGVIGPQREEFPRDLSLISPLAQARSSSRTPSDKPAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 6 - DNA, 696 bp, Artificial Sequence
  source: /note=“Description of Artificial Sequence Synthetic polynucleotide”
atgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcaagatcatcttctcgaaccccgagtgacaagcctgcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtcg

SEQ ID NO: 7 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 8 - protein, 157 aa, Homo sapiens [sequence omitted]

SEQ ID NO: 9 - protein, 113 aa, Unknown
  source: /note=“Description of Unknown DAP12 sequence”
MGGLEPCSRLLLLPLLLAVSGLRPVQAQAQSDCSCSTVSPGVLAGIVMGDLVLTVLIALAVYFLGRLVPRGRGAAEAATRKQRITETESPYQELQGQRSDVYSDLNTQRPYYK

SEQ ID NO: 10 - DNA, 342 bp, Unknown
  source: /note=“Description of Unknown DAP12 sequence”
atggggggacttgaaccctgcagcaggctcctgctcctgcctctcctgctggctgtaagtggtctccgtcctgtccaggcccaggcccagagcgattgcagttgctctacggtgagcccgggcgtgctggcagggatcgtgatgggagacctggtgctgacagtgctcattgccctggccgtgtacttcctgggccggctggtccctcgggggcgaggggctgcggaggcagcgacccggaaacagcgtatcactgagaccgagtcgccttatcaggagctccagggtcagaggtcggatgtctacagcgacctcaacacacagaggccgtattacaaatga

SEQ ID NO: 11 - protein, 182 aa, Unknown
  source: /note=“Description of Unknown CD28 sequence”
KFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRG

SEQ ID NO: 12 - DNA, 209 bp, Unknown
  source: /note=“Description of Unknown CD28 sequence”
attttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcacgcg

SEQ ID NO: 13 - protein, 247 aa, Unknown
  source: /note=“Description of Unknown IgG1 hinge sequence”
SYVTVSSQDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPK

SEQ ID NO: 14 - DNA, 734 bp, Unknown
  source: /note=“Description of Unknown IgG1 hinge sequence”
gtacgtcactgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatc

SEQ ID NO: 15 - DNA, 9764 bp, Artificial Sequence
  source: /note=“Description of Artificial Sequence Synthetic polynucleotide”
aatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtggggtacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatgctcgagatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcaagatcatcttctcgaaccccgagtgacaaggtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtcgcgagccgagggcaggggaagtcttctaacatgcggggacgtggaggaaaatcccgggcccatggagtttgggctgagctggctttttcttgtggctattttaaaaggtgtccagtgctctagagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagctgaaacgtggtggtggtggttctggtggtggtggttctggcggcggcggctccggtggtggtggatccgaggtgcagctgcagcagtctggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaagggaccacggtcactgtctcctcgtacgtcactgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctcacgcgtgaagttcagcaggagcgcagacgcccccgcgtaccagcagggccagaaccagctctataacgagctcaatctaggacgaagagaggagtacgatgttttggacaaaagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctgacaattgcgcgtcatcatcgatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgactgtgaaaatgtgtgtatgggtgtgtgtgaatgtgtgtatgtatgtgtgtgtgtgagtgtgtgtgtgtgtgtgtgcatgtgtgtgtgtgtgactgtgtctatgtgtatgactgtgtgtgtgtgtgtgtgtgtgtgtgtgtgtgtgtgtgtgtgtgtgttgtgaaaaaatattctatggtagtgagagccaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagcaagccagtttgcatctgtcaggatcaatttcccattatgccagtcatattaattactagtcaattagttgatttttatttttgacatatacatgtg

SEQ ID NO: 16 - DNA, 9452 bp, Artificial Sequence
  source: /note=“Description of Artificial Sequence Synthetic polynucleotide”
aatgaaagaccccacctgtaggtttggcaagctagcttaagtaacgccattttgcaaggcatggaaaaatacataactgagaatagaaaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttatgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcatttgggggctcgtccgggatcgggagacccctgcccagggaccaccgacccaccaccgggaggtaagctggccagcaacttatctgtgtctgtccgattgtctagtgtctatgactgattttatgcgcctgcgtcggtactagttagctaactagctctgtatctggcggacccgtggtggaactgacgagttcggaacacccggccgcaaccctgggagacgtcccagggacttcgggggccgtttttgtggcccgacctgagtcctaaaatcccgatcgtttaggactctttggtgcaccccccttagaggagggatatgtggttctggtaggagacgagaacctaaaacagttcccgcctccgtctgaatttttgctttcggtttgggaccgaagccgcgccgcgcgtcttgtctgctgcagcatcgttctgtgttgtctctgtctgactgtgtttctgtatttgtctgaaaatatgggcccgggctagcctgttaccactcccttaagtttgaccttaggtcactggaaagatgtcgagcggatcgctcacaaccagtcggtagatgtcaagaagagacgttgggttaccttctgctctgcagaatggccaacctttaacgtcggatggccgcgagacggcacctttaaccgagacctcatcacccaggttaagatcaaggtcttttcacctggcccgcatggacacccagaccaggtggggtacatcgtgacctgggaagccttggcttttgacccccctccctgggtcaagccctttgtacaccctaagcctccgcctcctcttcctccatccgccccgtctctcccccttgaacctcctcgttcgaccccgcctcgatcctccctttatccagccctcactccttctctaggcgcccccatatggccatatgagatcttatatggggcacccccgccccttgtaaacttccctgaccctgacatgacaagagttactaacagcccctctctccaagctcacttacaggctctctacttagtccagcacgaagtctggagacctctggcggcagcctaccaagaacaactggaccgaccggtggtacctcacccttaccgagtcggcgacacagtgtgggtccgccgacaccagactaagaacctagaacctcgctggaaaggaccttacacagtcctgctgaccacccccaccgccctcaaagtagacggcatcgcagcttggatacacgccgcccacgtgaaggctgccgaccccgggggtggaccatcctctagactgccatgctcgagatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcaagatcatcttctcgaaccccgagtgacaaggtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtcgcgagccgagggcaggggaagtcttctaacatgcggggacgtggaggaaaatcccgggcccatggagtttgggctgagctggctttttcttgtggctattttaaaaggtgtccagtgctctagagacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagctgaaacgtggtggtggtggttctggtggtggtggttctggcggcggcggctccggtggtggtggatccgaggtgcagctgcagcagtctggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggccaagggaccacggtcactgtctcctcgtacgtcactgtctcttcacaggatcccgccgagcccaaatctcctgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcaaccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaaaaagatcccaaattttggggcgtgctggccggaatcgtgatgggcgacctggtgctgacagtgctgatcgccctggctgtgtacttcctgggcagactggtgcccagaggaagaggcgctgccgaagccgccacccggaagcagagaatcaccgagacagagagcccctatcaggaactgcagggccagcggagcgacgtgtacagcgacctgaatacccagcggccctactacaaacgcggaccgcagtgtactaattatgctctcttgaaattggctggagatgttgagagcaatcccgggcccatgcgcattagcaagccccacctgcggagcatcagcatccagtgctacctgtgcctgctgctgaacagccacttcctgaccgaggccggcatccacgtgttcatcctgggctgcttcagcgccggactgcccaagaccgaggccaactgggtgaacgtgatcagcgacctgaagaagatcgaggacctgatccagagcatgcacatcgacgccaccctgtacaccgagagcgacgtgcaccccagctgcaaggtgaccgccatgaagtgctttctgctggaactgcaggtgatcagcctggaaagcggcgacgccagcatccacgacaccgtggagaacctgatcatcctggccaacaacagcctgagcagcaacggcaacgtgaccgagagcggctgcaaagagtgcgaggaactggaagagaagaacatcaaagagtttctgcagagcttcgtgcacatcgtgcagatgttcatcaacaccagctgacaattgcgcgtcatcatcgatccggattagtccaatttgttaaagacaggatatcagtggtccaggctctagttttgactcaacaatatcaccagctgaagcctatagagtacgagccatagataaaataaaagattttatttagtctccagaaaaaggggggaatgaaagaccccacctgtaggtttggcaagctagcttaagtaacgccattttgcaaggcatggaaaaatacataactgagaatagagaagttcagatcaaggtcaggaacagatggaacagctgaatatgggccaaacaggatatctgtggtaagcagttcctgccccggctcagggccaagaacagatggaacagctgaatatgggccaaacaggatatctgtggtaagcagttcctgccccggctcagggccaagaacagatggtccccagatgcggtccagccctcagcagtttctagagaaccatcagatgtttccagggtgccccaaggacctgaaatgaccctgtgccttatttgaactaaccaatcagttcgcttctcgcttctgttcgcgcgcttctgctccccgagctcaataaaagagcccacaacccctcactcggggcgccagtcctccgattgactgagtcgcccgggtacccgtgtatccaataaaccctcttgcagttgcatccgacttgtggtctcgctgttccttgggagggtctcctctgagtgattgactacccgtcagcgggggtctttcacacatgcagcatgtatcaaaattaatttggttttttttcttaagtatttacattaaatggccatagtacttaaagttacattggcttccttgaaataaacatggagtattcagaatgtgtcataaatatttctaattttaagatagtatctccattggctttctactttttcttttatttttttttgtcctctgtcttccatttgttgttgttgttgtttgtttgtttgtttgttggttggttggttaatttttttttaaagatcctacactatagttcaagctagactattagctactctgtaacccagggtgaccttgaagtcatgggtagcctgctgttttagccttcccacatctaagattacaggtatgagctatcatttttggtatattgattgattgattgattgatgtgtgtgtgtgtgattgtgtttgtgtgtgtgactgtgaaaatgtgtgtatgggtgtgtgtgaatgtgtgtatgtatgtgtgtgtgtgagtgtgtgtgtgtgtgtgtgcatgtgtgtgtgtgtgactgtgtctatgtgtatgactgtgtgtgtgtgtgtgtgtgtgtgtgtgtgtgtgtgtgtgtgtgtgttgtgaaaaaatattctatggtagtgagagccaacgctccggctcaggtgtcaggttggtttttgagacagagtctttcacttagcttggaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagctttgctcttaggagtttcctaatacatcccaaactcaaatatataaagcatttgacttgttctatgccctagggggcggggggaagctaagccagctttttttaacatttaaaatgttaattccattttaaatgcacagatgtttttatttcataagggtttcaatgtgcatgaatgctgcaatattcctgttaccaaagctagtataaataaaaatagataaacgtggaaattacttagagtttctgtcattaacgtttccttcctcagttgacaacataaatgcgctgctgagcaagccagtttgcatctgtcaggatcaatttcccattatgccagtcatattaattactagtcaattagttgatttttatttttgacatatacatgtg

SEQ ID NO: 17 - protein, 33 aa, Homo sapiens
MSTESMIRDVELAEEALPKKTGGPQGSRRCLFL

SEQ ID NO: 18 - protein, 4 aa, Homo sapiens
STES

SEQ ID NO: 19 - protein, 21 aa, Homo sapiens
FSFLIVAGATTLFCLLHFGVI

SEQ ID NO: 20 - protein, 20 aa, Homo sapiens
GPQREEFPRDLSLISPLAQA

SEQ ID NO: 21 - protein, 13 aa, Homo sapiens
VRSSSRTPSDKPV

SEQ ID NO: 22 - DNA, 662 bp, Artificial Sequence
  source: /note=“Description of Artificial Sequence Synthetic polynucleotide”
atgagcactgaaatgcatcccggaagggggtcctggcacgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgcg

SEQ ID NO: 23 - protein, 233 aa, Artificial Sequence
  source: /note=“Description of Artificial Sequence Synthetic polypeptide”
MSTEMHPGRGSWHEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 24 - DNA, 663 bp, Artificial Sequence
  source: /note=“Description of Artificial Sequence Synthetic polynucleotide”
atgagcactgaaagcaagatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcccaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtcg

SEQ ID NO: 25 - protein, 220 aa, Artificial Sequence
  source: /note=“Description of Artificial Sequence Synthetic polypeptide”
MSTESKIRDVELAEEALPKKTGGPQGSRRCLFLSLFSFLIVAGATTLFCLLHFGVIGPQREEFPRDLSLISPLAQAAHVVANPQAEGQLQWLNRRANALLANGVELRDNQLVVPSEGLYLIYSQVLFKGQGCPSTHVLLTHTISRIAVSHQTKVNLLSAIKSPCQRETPEGAEAKPWYEPIYLGGVFQLEKGDRLSAEINRPDYLDFAESGQVYFGIIAL

SEQ ID NO: 26 - DNA, 663 bp, Artificial Sequence
  source: /note=“Description of Artificial Sequence Synthetic polynucleotide”
atgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttcttcctgctgcactttggagtgatcggcccccagagggaagagttccccagggacctctctctaatcagccctctggcccaggcagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctcttcgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtcg

SEQ ID NO: 27 - protein, 220 aa, Artificial Sequence
  source: /note=“Description of Artificial Sequence Synthetic polypeptide”
MSTESMIRDVELAEEALPKKTGGPQGSRRCLFLSLFSFLIVAGATTLFFLLHFGVIGPQREEFPRDLSLISPLAQAAHVVANPQAEGQLQWLNRRANALLANGVELRDNQLVVPSEGLYLIYSQVLFKGQGCPSTHVLLTHTISRIAVSYQTKVNLLFAIKSPCQRETPEGAEAKPWYEPIYLGGVFQLEKGDRLSAEINRPDYLDFAESGQVYFGIIAL

SEQ ID NO: 28 - DNA, 663 bp, Artificial Sequence
  source: /note=“Description of Artificial Sequence Synthetic polynucleotide”
atgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcatcgctgagatcaatcggcccgactatctcgactttgccgagtatgggcaggtctactttgggatcattgccctgtcg

SEQ ID NO: 29 - protein, 220 aa, Artificial Sequence
  source: /note=“Description of Artificial Sequence Synthetic polypeptide”
MSTESMIRDVELAEEALPKKTGGPQGSRRCLFLSLFSFLIVAGATTLFCLLHFGVIGPQREEFPRDLSLISPLAQAAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLIAEINRPDYLDFAEYGQVYFGIIAL

SEQ ID NO: 30 - DNA, 663 bp, Artificial Sequence
  source: /note=“Description of Artificial Sequence Synthetic polynucleotide”
atgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctctactttgccgagtctgggcaggtctactttgggatcattgccctgtcg

SEQ ID NO: 31 - protein, 220 aa, Artificial Sequence
  source: /note=“Description of Artificial Sequence Synthetic polypeptide”
MSTESMIRDVELAEEALPKKTGGPQGSRRCLFLSLFSFLIVAGATTLFCLLHFGVIGPQREEFPRDLSLISPLAQAAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLYFAESGQVYFGIIAL

SEQ ID NO: 32 - DNA, 660 bp, Artificial Sequence
  source: /note=“Description of Artificial Sequence Synthetic polynucleotide”
atgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctgcaggcagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctctactttgccgagtctgggcaggtctactttgggatcattgccctgtcg

SEQ ID NO: 33 - protein, 219 aa, Artificial Sequence
  source: /note=“Description of Artificial Sequence Synthetic polypeptide”
MSTESMIRDVELAEEALPKKTGGPQGSRRCLFLSLFSFLIVAGATTLFCLLHFGVIGPQREEFPRDLSLISPLAQAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLYFAESGQVYFGIIAL

SEQ ID NO: 34 - DNA, 671 bp, Artificial Sequence
  source: /note=“Description of Artificial Sequence Synthetic polynucleotide”
atgctcgagtcgagatgagcactgaaatgcatcccggaagggggtcctggcacgaggaggcgctccccaagaagacaggggggccccagggctccaggcggtgcttgttcctcagcctcttctccttcctgatcgtggcaggcgccaccacgctcttcttcctgctgcactttggagtgatcggcccccagagggaagagttccccagggacctctctctaatcagccctctggcagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcccaccagaccaaggtcaacctcctcttcgccatcaagagcccctgccagagggagaccccagagggggctgaggccaagccctggtatgagcccatctatctgggaggggtcttccagctggagaagggtgaccgactcatcgctgagatcaatcggcccgactatctctactttgccgagtatgggcaggtctactttgggatcattgccctgtcg

SEQ ID NO: 35 - protein, 219 aa, Artificial Sequence
  source: /note=“Description of Artificial Sequence Synthetic polypeptide”
MSTEMHPGRGSWHEEALPKKTGGPQGSRRCLFLSLFSFLIVAGATTLFFLLHFGVIGPQREEFPRDLSLISPLAQAHVVANPQAEGQLQWLNRRANALLANGVELRDNQLVVPSEGLYLIYSQVLFKGQGCPSTHVLLTHTISRIAVSHQTKVNLLFAIKSPCQRETPEGAEAKPWYEPIYLGGVFQLEKGDRLIAEINRPDYLYFAEYGQVYFGIIAL

SEQ ID NO: 36 - protein, 1131 aa, Artificial Sequence
  source: /note=“Description of Artificial Sequence Synthetic polypeptide”
MSTESMIRDVELAEEALPKKTGGPQGSRRCLFLSLFSFLIVAGATTLFCLLHFGVIGPQREEFPRDLSLISPLAQARSSSRTPSDKVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALSRAEGRGSLLTCGDVEENPGPMEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSSYVTVSSQDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 37 - protein, 1138 aa, Artificial Sequence
  source: /note=“Description of Artificial Sequence Synthetic polypeptide”
MSTESMIRDVELAEEALPKKTGGPQGSRRCLFLSLFSFLIVAGATTLFCLLHFGVIGPQREEFPRDLSLISPLAQARSSSRTPSDKVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIALSRAEGRGSLLTCGDVEENPGPMEFGLSWLFLVAILKGVQCSRDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLELKRGGGGSGGGGSGGGGSGGGGSEVQLQQSGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTTVTVSSYVTVSSQDPAEPKSPDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKKDPKFWGVLAGIVMGDLVLTVLIALAVYFLGRLVPRGRGAAEAATRKQRITETESPYQELQGQRSDVYSDLNTQRPYYKRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGPQCTNYALLKLAGDVESNPGPMRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 38 - DNA, 673 bp, Artificial Sequence
  source: /note=“Description of Artificial Sequence Synthetic polynucleotide”
tcgagtcgagatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctgcaggcagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtcgtcg

SEQ ID NO: 39 - protein, 219 aa, Artificial Sequence
  source: /note=“Description of Artificial Sequence Synthetic polypeptide”
MSTESMIRDVELAEEALPKKTGGPQGSRRCLFLSLFSFLIVAGATTLFCLLHFGVIGPQREEFPRDLSLISPLQAAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 40 - DNA, 654 bp, Artificial Sequence
  source: /note=“Description of Artificial Sequence Synthetic polynucleotide”
atgagcactgaaatgcatcccggaagggggtcctggcacgaggaggcgctccccaagaagacaggggggccccagggctccaggcggtgcttgttcctcagcctcttctccttcctgatcgtggcaggcgccaccacgctcttcttcctgctgcactttggagtgatcggcccccagagggaagagttccccagggacctctctctaatcagccctctggcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcccaccagaccaaggtcaacctcctcttcgccatcaagagcccctgccagagggagaccccagagggggctgaggccaagccctggtatgagcccatctatctgggaggggtcttccagctggagaagggtgaccgactcatcgctgagatcaatcggcccgactatctctactttgccgagtatgggcaggtctactttgggatcattgccctgtcg

SEQ ID NO: 41 - protein, 218 aa, Artificial Sequence
  source: /note=“Description of Artificial Sequence Synthetic polypeptide”
MSTEMHPGRGSWHEEALPKKTGGPQGSRRCLFLSLFSFLIVAGATTLFFLLHFGVIGPQREEFPRDLSLISPLAHVVANPQAEGQLQWLNRRANALLANGVELRDNQLVVPSEGLYLIYSQVLFKGQGCPSTHVLLTHTISRIAVSHQTKVNLLFAIKSPCQRETPEGAEAKPWYEPIYLGGVFQLEKGDRLIAEINRPDYLYFAEYGQVYFGIIALS

SEQ ID NO: 42 - DNA, 33 bp, Artificial Sequence
  source: /note=“Description of Artificial Sequence Synthetic primer”
tcgagaagatgatctgactgcctgggccagagg

SEQ ID NO: 43 - DNA, 30 bp, Artificial Sequence
  source: /note=“Description of Artificial Sequence Synthetic primer”
tcgagaagatgatcttgcctgggccagagg

SEQ ID NO: 44 - DNA, 12 bp, Artificial Sequence
  source: /note=“Description of Artificial Sequence Synthetic oligonucleotide”
tgatcttgcctg

SEQ ID NO: 45 - DNA, 33 bp, Artificial Sequence
  source: /note=“Description of Artificial Sequence Synthetic primer”
tacaacatgggctacaggcttgtcactcggggt

SEQ ID NO: 46 - DNA, 30 bp, Artificial Sequence
  source: /note=“Description of Artificial Sequence Synthetic primer”
tacaacatgggctaccttgtcactcggggt

SEQ ID NO: 47 - DNA, 12 bp, Artificial Sequence
  source: /note=“Description of Artificial Sequence Synthetic oligonucleotide”
ggctaccttgtc

SEQ ID NO: 48 - DNA, 48 bp, Artificial Sequence
  source: /note=“Description of Artificial Sequence Synthetic oligonucleotide”
caggcagtcagatcatcttctcgaaccccgagtgacaagcctgtagcc

SEQ ID NO: 49 - protein, 16 aa, Artificial Sequence
  source: /note=“Description of Artificial Sequence Synthetic peptide”
QAVRSSSRTPSDKPVA

SEQ ID NO: 50 - protein, 220 aa, Unknown; identical to SEQ ID NO: 3
  source: /note=“Description of Unknown TNF alpha sequence”

SEQ ID NO: 51 - protein, 233 aa, Homo sapiens; identical to SEQ ID NO: 7

SEQ ID NO: 52 - DNA, 660 bp, Artificial Sequence
  source: /note=“Description of Artificial Sequence Synthetic polynucleotide”
  misc_feature (214)..(219): /note=“This region may or may not be present”
atgagcactgaaatgcatcccggaagggggtcctggcacgaggaggcgctccccaagaagacaggggggccccagggctccaggcggtgcttgttcctcagcctcttctccttcctgatcgtggcaggcgccaccacgctcttcttcctgctgcactttggagtgatcggcccccagagggaagagttccccagggacctctctctaatcagccctctggccgcagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcccaccagaccaaggtcaacctcctcttcgccatcaagagcccctgccagagggagaccccagagggggctgaggccaagccctggtatgagcccatctatctgggaggggtcttccagctggagaagggtgaccgactcatcgctgagatcaatcggcccgactatctctactttgccgagtatgggcaggtctactttgggatcattgccctgtcg

SEQ ID NO: 53 - protein, 220 aa, Artificial Sequence
  source: /note=“Description of Artificial Sequence Synthetic polypeptide”
  SITE (74)..(75): /note=“This region may or may not be present”
MSTEMHPGRGSWHEEALPKKTGGPQGSRRCLFLSLFSFLIVAGATTLFFLLHFGVIGPQREEFPRDLSLISPLAQAHVVANPQAEGQLQWLNRRANALLANGVELRDNQLVVPSEGLYLIYSQVLFKGQGCPSTHVLLTHTISRIAVSHQTKVNLLFAIKSPCQRETPEGAEAKPWYEPIYLGGVFQLEKGDRLIAEINRPDYLYFAEYGQVYFGIIALS

SEQ ID NO: 54 - protein, 56 aa, Unknown
  source: /note=“Description of Unknown monkey TNF alpha sequence”
MSTESMIRDVELAEEALPRKTAGPQGSRRCWFLSLFSFLLVAGATTLFCLLHFGVI

SEQ ID NO: 55 - protein, 56 aa, Sus sp.
MSTESMIRDVELAEEALAKKAGGPQGSRRCLCLSLFSFLLVAGATTLFCLLHFEVI

SEQ ID NO: 56 - protein, 56 aa, Mus sp.
MSTESMIRDVELAEEALPQKMGGFQNSRRCLCLSLFSFLLVAGATTLFCLLNFGVI

SEQ ID NO: 57 - protein, 56 aa, Oryctolagus sp.
MSTESMIRDVELAEGPLPKKAGGPQGSKRCLCLSLFSFLLVAGATTLFCLLHFRVI

SEQ ID NO: 58 - protein, 56 aa, Capra sp.
MSTKSMIRDVELAEEVLSKKAGGPQGSRSCWCLSLFSFLLVAGATTLFCLLHFGVI

SEQ ID NO: 59 - protein, 56 aa, Ovis sp.
MSTKSMIRDVELAEEVLSNKAGGPQGSRSCWCLSLFSFLLVAGATTLFCLLHFGVI

(19) United States
(12) Patent Application Publication
(10) Pub. No.: US 2022/0023343 A1
(43) Pub. Date: Jan. 27, 2022

(54) SUICIDE GENE

(71) Applicant: Board of Regents, The University of Texas System, Austin, TX (US)

(72) Inventors: Katy Rezvani, Houston, TX (US); Elizabeth Shpall, Houston, TX (US)

(21) Appl. No.: 17/309,315

(22) PCT Filed: Nov. 18, 2019
(86) PCT No.: PCT/US2019/062009

§ 371 Date: May 18, 2021

Related U.S. Application Data
(60) Provisional application No. 62/769,405, filed on Nov. 19, 2018.
(60) Provisional application No. 62/773,372, filed on Nov. 30, 2018.
(60) Provisional application No. 62/791,464, filed on Jan. 11, 2019.

(51) Int. Cl. A61K 35/17; C07K 14/725; C07K 14/525

(52) U.S. Cl. CPC A61K 35/17; A61K 38/00; C07K 14/525; C07K 14/7051

(57) ABSTRACT

Embodiments of the disclosure encompass particular TNF-alpha mutants that are nonsecretable and membrane bound, thereby providing a target for inhibition in cells that express the mutants. In specific embodiments, the TNF-alpha mutants are utilized as a suicide gene in cells employed for adoptive cell therapy for an individual, wherein at a desired time the individual is provided one or more anti-TNF-alpha antibodies that bind the membrane bound TNF-alpha and elicit complement-dependent cytotoxicity for the cells. The TNF-alpha mutant can also be used as a way of tracking the transduced cells in vivo.

The present application claims priority to U.S. Provisional Patent Application Ser. No. 62/769,405, filed Nov. 19, 2018; U.S. Provisional Patent Application Ser. No. 62/773,372, filed Nov. 30, 2018; and U.S. Provisional Patent Application Ser. No. 62/791,464, filed Jan. 11, 2019, all of which applications are incorporated by reference herein in their entirety.

### SEQUENCE LISTING

The instant application contains a Sequence Listing which has been submitted electronically in ASCII format and is hereby incorporated by reference in its entirety. Said ASCII copy, created on Nov. 13, 2019, is named UTFC_P1151WO_SL.txt and is 108,130 bytes in size.

### TECHNICAL FIELD

Embodiments of the disclosure encompass at least the fields of cell biology, molecular biology, immunology, cell therapy, and medicine.

### BACKGROUND

Adoptive cell therapy with chimeric antigen receptor (CAR)-engineered and T-cell receptor (TCR)-transduced T cells has been associated with reports of serious adverse events such as cytokine release syndrome and neurotoxicity, as well as on-target/off tumor toxicities. As increasing numbers of patients are treated with adoptive cell therapy, the incorporation of a safety mechanism to allow selective deletion of the adoptively infused cells in the face of toxicity is useful.

The present disclosure provides a solution for a long-felt need in the art of safety mechanisms for cell therapies.

### BRIEF SUMMARY

Embodiments of the present disclosure are directed to systems, methods, and compositions related to cell therapy, including safety mechanisms to control cell therapy. In particular embodiments, a unique suicide gene is utilized in conjunction with cell therapy of any kind to control its use and allow for controllable termination of the cell therapy at a desired event and/or time. The suicide gene is employed in transduced cells for the purpose of eliciting death for the transduced cells when needed. In specific embodiments, the suicide/depletion gene is a tumor necrosis factor (TNF)-alpha mutant that is uncleavable by standard enzymes that cleave TNF in nature, such as TNF-alpha-converting enzyme (also referred to as TACE). As such, the TNF-alpha mutant is membrane-bound, inactive, and nonsecretable, in particular embodiments. The TNF-alpha mutant of the disclosure is targetable by one or more agents that bind the mutant, including at least an antibody, such that following binding of the agent(s) to the TNF-alpha mutant on the surface of the cell, the cell dies. Embodiments of the disclosure allow the TNF-alpha mutant to be utilized as a marker for cells that express it.

Embodiments of the disclosure include compositions comprising a transduced cell comprising a nucleic acid that encodes one or more engineered nonsecretable tumor necrosis factor (TNF)-alpha mutant polypeptides and a nucleic acid that encodes one or more therapeutic gene products. In specific embodiments, the TNF-alpha mutant polypeptide comprises a deletion with respect to SEQ ID NO:8 of the following: amino acid residue 1 and amino acid residue 12; amino acid residue 1 and amino acid residue 13; amino acid residues 1-12; amino acid residues 1-13; or amino acid residues 1-14. The therapeutic gene product of the composition may or may not be an engineered receptor, such as a T-cell receptor, a chimeric antigen receptor (CAR), cytokine receptor, homing receptor, or chemokine receptor. Any of the engineered receptors may or may not target an antigen, such as a cancer antigen. When the engineered receptor is a CAR, the CAR may or may not comprises one or more costimulatory domains, such as CD28, DAP12, or both.

In particular embodiments, the nucleic acid that encodes the TNF-alpha mutant polypeptide and the nucleic acid that encodes the therapeutic gene product are the same nucleic acid molecule, although the nucleic acid that encodes the TNF-alpha mutant polypeptide and the nucleic acid that encodes the therapeutic gene product may be different nucleic acid molecules. In any case, the nucleic acid molecule may be a vector, including a viral vector (retroviral vector, lentiviral vector, adenoviral vector, or adeno-associated viral vector) or a non-viral vector, such one that comprises a plasmid, lipid, transposon, or combination thereof.

The transduced cells of the composition of the disclosure may be an immune cell or a stem cell, for example. Examples of an immune cell includes a T cell, a NK cell, NKT cell, iNKT cell, B cell, regulatory T cell, monocyte, macrophage, dendritic cell, or mesenchymal stromal cell. The cell may or may not express one or more exogenously provided cytokines, such as IL-15, IL-12, IL-18, IL-21 or combination thereof. The cytokine may or may not be encoded from the same vector as the TNF-alpha mutant gene. In specific cases, the cytokine is expressed as a separate polypeptide molecule as the TNF-alpha mutant and as an engineered receptor of the cell.

In particular embodiments of the disclosure, the TNF-alpha mutant polypeptide comprises SEQ ID NO:1, SEQ ID NO:3, SEQ ID NO:5, or SEQ ID NO:39. The TNF-alpha mutant polypeptide may be encoded by a sequence that comprises SEQ ID NO:2, SEQ ID NO:4, SEQ ID NO:6, or SEQ ID NO:38. In certain aspects, the TNF-alpha mutant polypeptide lacks one or more further mutations that prevent binding of the TNF-alpha mutant polypeptide to a TNF receptor.

Embodiments of the disclosure include methods of inducing death for a transduced cell expressing at least an engineered nonsecretable TNF-alpha mutant polypeptide and optionally expressing a therapeutic gene, such as an engineered receptor, the methods comprising the step of providing an effective amount of at least one agent that binds the TNF-alpha mutant on the transduced cell. An agent that binds TNF-alpha may be an antibody, small molecule, polypeptide, nucleic acid, or combination thereof, for example. When the agent is an antibody, the antibody may be of any kind, including at least a monoclonal antibody. In the methods, the cell may further express an engineered receptor, including a T-cell receptor, a chimeric antigen receptor (CAR), cytokine receptor, homing receptor, or chemokine receptor. Any of the engineered receptors may bind a cancer or other antigen. In specific cases, the method occurs in vivo in an individual with a medical condition and when the individual has been provided a therapy for the medical condition that comprises a plurality of the transduced cells. Although the medical condition may be of any kind, in specific cases the medical condition is cancer. The agent may be provided to the individual upon onset of one or more adverse events from the therapy or when an adverse event is suspected of occurring. The individual may exhibit one or more symptoms of cytokine release syndrome, neurotoxicity, anaphylaxis/allergy, and/or on-target/off tumor toxicity. In some cases, the individual has been provided, is provided, and/or will be provided an additional therapy for the medical condition. In particular aspects of the disclosure, the TNF-alpha mutant polypeptide lacks or comprises one or more further mutations that prevent binding of the TNF-alpha mutant polypeptide to a TNF receptor or prevents reverse signaling.

Embodiments of the disclosure include methods of reducing the effects of cytokine release syndrome in an individual that has received and/or who is receiving cell therapy with cells that express a nonsecretable TNF-alpha mutant, comprising the step of providing an effective amount of one or more agents that bind the mutant to cause in the individual (a) elimination of at least some of the cells of the cell therapy; and (b) reduction in the level of soluble TNF-alpha.

Embodiments of the disclosure include methods of reducing the risk of toxicity of a cell therapy for an individual, comprising the step of modifying the cells of the cell therapy to express a nonsecretable TNF-alpha mutant. The cell therapy may be for cancer, for example. The cell therapy may comprise an engineered receptor that targets an antigen.

Specific embodiments include vectors comprising a sequence that encodes a nonsecretable TNF-alpha mutant and that encodes an engineered receptor. The nonsecretable TNF-alpha mutant and the engineered receptor may or may not be encoded from the vector as separate polypeptides. In specific cases, sequence of the vector that encodes the nonsecretable TNF-alpha mutant and sequence of the vector that encodes the engineered receptor are separated on the vector by a 2A element or an IRES element. The vector may or may not further encode a cytokine, such as IL-15, IL-12, IL-18, IL-2, IL-7, or IL-21. The cytokine may be expressed from the vector as a separate polypeptide as the TNF-alpha mutant and the engineered receptor.

Embodiments of the disclosure include compositions of matter including a nucleic acid sequence comprising SEQ ID NO:15 or SEQ ID NO:16.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention. Aspects of an embodiment set forth in the Examples are also embodiments that may be implemented in the context of embodiments discussed elsewhere in a different Example or elsewhere in the application, such as in the Brief Summary, Detailed Description, Claims, and Brief Description of Drawings.

The foregoing has outlined rather broadly the features and technical advantages of the present disclosure in order that the detailed description that follows may be better understood. Additional features and advantages will be described hereinafter which form the subject of the claims herein. It should be appreciated by those skilled in the art that the conception and specific embodiments disclosed may be readily utilized as a basis for modifying or designing other structures for carrying out the same purposes of the present designs. It should also be realized by those skilled in the art that such equivalent constructions do not depart from the spirit and scope as set forth in the appended claims. The novel features which are believed to be characteristic of the designs disclosed herein, both as to the organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

### BRIEF DESCRIPTION OF THE DRAWINGS

For a more complete understanding of the present disclosure, reference is now made to the following descriptions taken in conjunction with the accompanying drawings.

FIG. 1 is one example of an experimental plan to mutagenize TNF-alpha in order to ablate membrane cutting sites. Perez et al. (1990) reported that deletion in Valine at positions 1 and Proline at position 12 of the extracellular portion of TNF-alpha results in biologically active but non-cleavable TNF-alpha. The underlined nucleotides in the left panel show the deleted nucleotides during mutagenesis corresponding to positions 229-279 of nucleotide sequence. The wild type primer TCGAGAAGATGATCTGACTGCCTGGGCCAGAGG is SEQ ID NO:42, the Del VAL1 mutant primer TCG AGA AGA TGA TCT TGC CTG GGC CAG AGG-3 is SEQ ID NO:43, and the CP496 oligonucleotide TGA TCT TGC CTG is SEQ ID NO:44. The wild type primer TAC AAC ATG GGC TACAGGCTTGTCACTCGGGGT is SEQ ID NO:45, the Del PRO 12 mutant primer TAC AAC ATG GGC TAC CTT GTC ACT CGG GGT is SEQ ID NO:46, and the CP498 oligonucleotide GGC TAC CTT GTC is SEQ ID NO:47. The Perez et al. (1990) sequence CAGGCAGTCAGATCATCTTCTCGAACCCCGAGTGACAAGCCTGTAGCC is SEQ ID NO:48, and the sequence QAVRSSSRTPSDKPVA is SEQ ID NO:49.

FIG. 2A illustrates one example of a vector that separately encodes a TNF-alpha mutant (delVal1 and delProl12) and an example of a CD19-specific chimeric antigen receptor (CAR) (left panel). The right panel illustrates an example of a vector configuration in which the mutant TNF-alpha is encoded as a separate polypeptide from both the CAR molecule and a cytokine. FIG. 2B illustrates one example of a vector that separately encodes a TNF-alpha mutant (delVal13), an example of a CAR, and a cytokine. FIG. 2C illustrates one example of a vector that separately encodes a TNF-alpha mutant (delVal1 and delVal13) and an example of a CAR. FIG. 2D illustrates one example of a vector that separately encodes a TNF-alpha mutant (where 13 aa spanning Val 1 to Val 13 have been deleted) and an example of a CAR. FIG. 2E illustrates one example of a vector that separately encodes a TNF-alpha mutant (delAla-1 to delVal13 where 14 aa spanning from Ala-1 to Val13 have been deleted) and an example of a CAR.

FIG. 3 shows that NK cells transduced with a vector having a construct separately encoding both a TNF-alpha mutant and a CAR express both the CAR and TNF-alpha on their surface.

FIG. 4A illustrates examples of TNF-alpha inhibitors.

FIG. 4B demonstrates that NK cells transduced with a vector having a construct separately encoding both a TNF-alpha mutant and a CAR are targeted by TNF-alpha antagonists and eliminated by complement-dependent cytotoxicity (CDC). Greater than 70% of NK cells expressing mutant TNF-alpha are eliminated by CDC within 90 minutes of treatment with infliximab.

FIG. 5A shows that NK cells transduced with a vector having a construct separately encoding both a TNF-alpha mutant and a CAR produce more effector cytokines and degranulate more efficiently than CAR19-NK cells in response to Raji targets. FIG. 5B shows NK cells transduced with a vector having a construct separately encoding both a TNF-alpha mutant and a CAR construct kill Raji targets efficiently.

FIG. 6 demonstrates that NK cells transduced with a vector that separately expresses a CD19-specific CAR and a TNF-alpha mutant do not exhibit off-target activity.

FIG. 7 demonstrates that NK cells transduced with a vector that separately expresses a CD19-specific CAR and a TNF-alpha mutant do not exhibit off-target activity and do not secrete TNF-alpha non-specifically.

FIG. 8 illustrates that TNF-alpha receptor binding sites for TNF receptors 1 and 2 vs. TNF-alpha antibodies infliximab and adalimumab are different. The sequence in the figure is SEQ ID NO:50.

FIG. 9 provides a structure of TNF-alpha with noted domains. The sequences in the figure are SEQ ID NOS 17, 54-59, 51, 18, and 18-21, respectively, in order of appearance.

FIG. 10 illustrates a TNFalpha mutation that combines a mutation in the casein kinase I (CKI) consensus sequence in the cytoplasmic domain (underlined) with deletion of Ala-3 and Gln −2 (in addition to deletion of Ala −1 through and including deletion of Val13 that is not depicted) in addition to six examples of additional mutations that interfere with binding to TNF Receptor 1 and TNF Receptor 2 (such mutated sequences are double underlined). The nucleotide sequence in the figure is SEQ ID NO:52, and the polypeptide sequence in the figure is SEQ ID NO:53.

FIGS. 11A-11B demonstrate that antitumor activity of NK cells transduced with a TNF-alpha mutant-CAR19-IL15 construct is superior to the iC9-CAR19-IL15 construct. In FIG. 11A, NSG mice with Raji tumor received 3×10e6 CAR cord blood NK cells transduced with TNF-alpha mut-CAR19-IL15 construct or iC9-CAR19-IL15 construct. FIG. 11B demonstrates percent survival over time.

### DETAILED DESCRIPTION

The present disclosure incorporates by reference herein U.S. Provisional Patent Application Ser. No. 62/769,414, filed Nov. 19, 2018; U.S. Provisional Patent Application Ser. No. 62/773,394, filed Nov. 30, 2018; and U.S. Provisional Patent Application Ser. No. 62/791,491, filed Jan. 11, 2019.

As used herein the specification, “a” or “an” may mean one or more. As used herein in the claim(s), when used in conjunction with the word “comprising”, the words “a” or “an” may mean one or more than one. As used herein “another” may mean at least a second or more. In specific embodiments, aspects of the disclosure may “consist essentially of” or “consist of” one or more sequences of the disclosure, for example. Some embodiments of the invention may consist of or consist essentially of one or more elements, method steps, and/or methods of the disclosure. It is contemplated that any method or composition described herein can be implemented with respect to any other method or composition described herein. The scope of the present application is not intended to be limited to the particular embodiments of the process, machine, manufacture, composition of matter, means, methods and steps described in the specification. As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

The term “engineered” as used herein refers to an entity that is generated by the hand of man, including a cell, nucleic acid, polypeptide, vector, and so forth. In at least some cases, an engineered entity is synthetic and comprises elements that are not naturally present or configured in the manner in which it is utilized in the disclosure.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

### I. General Embodiments

Embodiments of the present disclosure concern methods and compositions that provide for a cell therapy to be terminated. The present disclosure provides both a marker moiety and a suicide/depletion moiety for cell therapy, based on uncleavable mutants of the 26 kd TNF-alpha. The TNF-alpha mutants are uncleavable that leaves them membrane bound and nonsecretable. Cells expressing the uncleavable TNF-alpha mutants can be targeted for selective deletion including, for example, using FDA-approved TNF-α antibodies currently in the clinic, such as etanercept, infliximab, or adalilumab. The mutated TNF-alpha polypeptide may be co-expressed with one or more therapeutic transgenes, such as a gene encoding a TCR or CAR. In addition, the TNF-alpha mutant expressing cells have superior activity against the tumor target, mediated by the biological activity of the membrane-bound TNF-alpha protein.

### II. TNF-Alpha Mutants

The present disclosure encompasses mutants of TNF-alpha whose expression in particular cells allows the mutant TNF to be targeted by an agent that binds the mutant, thereby causing death for the particular TNF-alpha mutant-bearing cells. In particular embodiments, the mutant TNF-alpha polypeptides are uncleavable and nonsecretable because of one or more mutations, and such uncleavable and nonsecretable polypeptides render the mutant TNF-alpha to be membrane bound. The association of the membrane bound TNF-alpha in the cell allows the cell to be killed when the membrane bound TNF-alpha is targeted by an agent that binds it directly or indirectly, including an inhibitor. In embodiments wherein the TNF-alpha inhibitor is an antibody, the cell may die by complement-dependent cytotoxicity, and in embodiments wherein the TNF-alpha inhibitor is not an antibody, the cell may die by another mechanism, such as apoptosis.

Therefore, in specific embodiments of the mutant, the membrane cutting site(s) are ablated, thereby retaining surface expression on the cell and endowing the ability of the cell to be targeted for destruction. Thus, the disclosure contemplates mutant membrane-bound TNF-alpha as a suicide gene for the selective deletion of transduced cells.

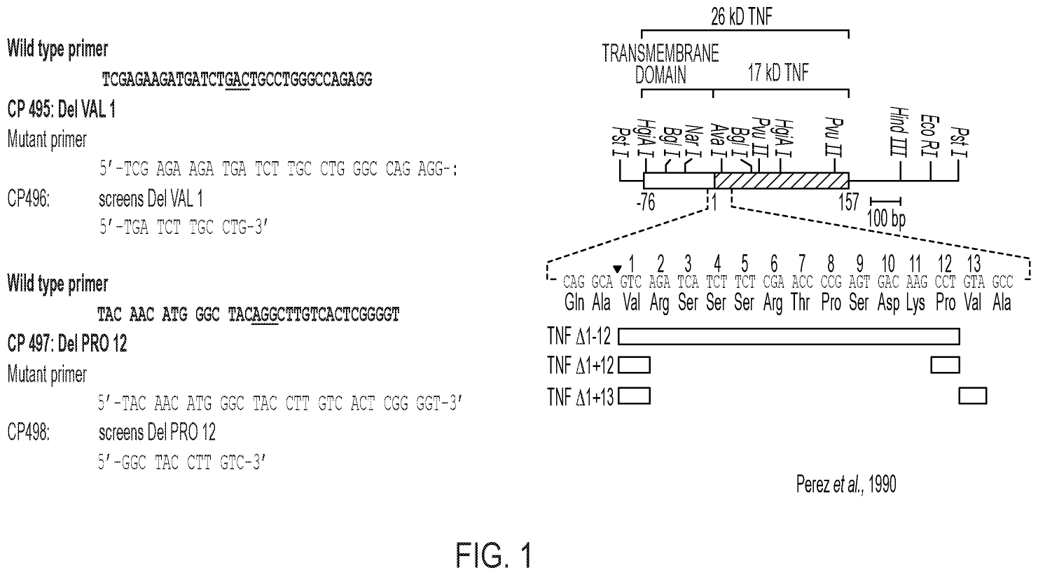

TNF-alpha has a 26 kD transmembrane form and a 17 kD secretory component. FIG. 1 herein (right panel from Perez et al. (1990)) illustrates some mutants encompassed by the disclosure. In specific embodiments, examples of TNF-alpha mutants of the disclosure include at least the following with respect to the 17 kD TNF: (1) deletion of Val1 and deletion of Pro112; (2) deletion of Val13; (3) deletion of Val1 and deletion of Val13; (4) deletion of Val1 through and including Pro112 and also deletion of Val13 (delete 13aa); (5) deletion of Ala −3 through to and including Val 13 (delete 16 aa); (6) deletion of Ala-1 through to and include Val13 (delete 14aa). In specific embodiments, a TNF-alpha mutant comprises deletion of the respective amino acid at position −3, −2, −1, 1, 2, 3, 4, 5, 6, 7, 8, 9, 10, 11, 12, 13, or a combination thereof. Specific combinations include deletions at positions −3 through and including 13; −3 through and including 12; −3 through and including 11; −3 through and including 10; −3 through and including 9; −3 through and including 8; −3 through and including 7; −3 through and including 6; −3 through and including 5; −3 through and including 4; −3 through and including 3; −3 through and including 2; −3 through and including 1; −3 through and including −1; −3 through and including −2; −2 through and including 13; −2 through and including 12; −2 through and including 11; −2 through and including 10; −2 through and including 9; −2 through and including 8; −2 through and including 7; −2 through and including 6; −2 through and including 5; −2 through and including 4; −2 through and including 3; −2 through and including 2; −2 through and including 1; −2 through and including −1; −1 through and including 13; −1 through and including 12; −1 through and including 11; −1 through and including 10; −1 through and including 9; −1 through and including 8; −1 through and including 7; −1 through and including 6; −1 through and including 5; −1 through and including 4; −1 through and including 3; −1 through and including 2; −1 through and including 1; 1 through and including 13; 1 through and including 12; 1 through and including 11; 1 through and including 10; 1 through and including 9; 1 through and including 8; 1 through and including 7; 1 through and including 6; 1 through and including 5; 1 through and including 4; 1 through and including 3; 1 through and including 2; and so forth.

The TNF-alpha mutants may be generated by any suitable method, but in specific embodiments they are generated by site-directed mutagenesis. In some cases, the TNF-alpha mutants may have mutations other than those that render the protein uncleavable. In specific cases, the TNF-alpha mutants may have 1, 2, 3, or more mutations other than the deletions at Val1, Pro12, and/or Val13 or the region there between. The mutations other than those that render the mutants nonsecretable may be one or more of an amino acid substitution, deletion, addition, inversion, and so forth. In cases wherein the additional mutation is an amino acid substitution, the substitution may or may not be to a conservative amino acid, for example. In some cases, 1, 2, 3, 4, 5, or more additional amino acids may be present on the N-terminal and/or C-terminal ends of the protein. In some cases, a TNF-alpha mutant has (1) one or more mutations that render the mutant nonsecretable; (2) one or more mutations that prevents outside-in signaling for the mutant; and/or (3) one or more mutations that interfere with binding of the mutant to TNF Receptor 1 and/or TNF Receptor 2 and render them inactive.

TNF-Alpha Mutant delVal1 delProl12 Amino Acid Sequence

(SEQ ID NO: 1)
MSTESMIRDVELAEEALPKKTGGPQGSRRCLFLSLFSFLIVAGATTLFCL
LHFGVIGPQREEFPRDLSLISPLAQARSSSRTPSDKVAHVVANPQAEGQL
QWLNRRANALLANGVELRDNQLVVPSEGLYLIYSQVLFKGQGCPSTHVLL
THTISRIAVSYQTKVNLLSAIKSPCQRETPEGAEAKPWYEPIYLGGVFQL
EKGDRLSAEINRPDYLDFAESGQVYFGIIAL

TNF-Alpha Mutant-delVal1 Del Pro112 Nucleic Acid Sequence

(SEQ ID NO: 2)
atgagcactgaaagcatgatccgggacgtggagctggccgaggaggcgct
ccccaagaagacaggggggccccagggctccaggcggtgcttgttcctca
gcctcttctccttcctgatcgtggcaggcgccaccacgctcttctgcctg
ctgcactttggagtgatcggcccccagagggaagagttccccagggacct
ctctctaatcagccctctggcccaggcaagatcatcttctcgaaccccga
gtgacaaggtagcccatgttgtagcaaaccctcaagctgaggggcagctc
cagtggctgaaccgccgggccaatgccctcctggccaatggcgtggagct
gagagataaccagctggtggtgccatcagagggcctgtacctcatctact
cccaggtcctcttcaagggccaaggctgcccctccacccatgtgctcctc
acccacaccatcagccgcatcgccgtctcctaccagaccaaggtcaacct
cctctctgccatcaagagcccctgccagagggagaccccagagggggctg
aggccaagccctggtatgagcccatctatctgggaggggtcttccagctg
gagaagggtgaccgactcagcgctgagatcaatcggcccgactatctcga
ctttgccgagtctgggcaggtctactttgggatcattgccctgtcg

TNFa Mutant-Del Val1 to Val13 Amino Acid Sequence (Delete 13Aa)

(SEQ ID NO: 3)
MSTESMIRDVELAEEALPKKTGGPQGSRRCLFLSLFSFLIVAGATTLFCL
LHFGVIGPQREEFPRDLSLISPLAQAAHVVANPQAEGQLQWLNRRANALL
ANGVELRDNQLVVPSEGLYLIYSQVLFKGQGCPSTHVLLTHTISRIAVSY
QTKVNLLSAIKSPCQRETPEGAEAKPWYEPIYLGGVFQLEKGDRLSAEIN
RPDYLDFAESGQVYFGIIAL

TNFa Mutant-Del Val1 to Pro112 delVal13 (Delete 13 Aa) Nucleic Acid Sequence

(SEQ ID NO: 4)
atgagcactgaaagcatgatccgggacgtggagctggccgaggaggcgct
ccccaagaagacaggggggccccagggctccaggcggtgcttgttcctca
gcctcttctccttcctgatcgtggcaggcgccaccacgctcttctgcctg
ctgcactttggagtgatcggcccccagagggaagagttccccagggacct
ctctctaatcagccctctggcccaggcagcccatgttgtagcaaaccctc
aagctgaggggcagctccagtggctgaaccgccgggccaatgccctcctg
gccaatggcgtggagctgagagataaccagctggtggtgccatcagaggg
cctgtacctcatctactcccaggtcctcttcaagggccaaggctgcccct
ccacccatgtgctcctcacccacaccatcagccgcatcgccgtctcctac
cagaccaaggtcaacctcctctctgccatcaagagcccctgccagaggga
gaccccagagggggctgaggccaagccctggtatgagcccatctatctgg
gaggggtcttccagctggagaagggtgaccgactcagcgctgagatcaat
cggcccgactatctcgactttgccgagtctgggcaggtctactttgggat
cattgccctgtcg

TNF-Alpha delVal1 delVal13 Amino Acid Sequence

(SEQ ID NO: 5)
MSTESMIRDVELAEEALPKKTGGPQGSRRCLFLSLFSFLIVAGATTLFCL
LHFGVIGPQREEFPRDLSLISPLAQARSSSRTPSDKPAHVVANPQAEGQL
QWLNRRANALLANGVELRDNQLVVPSEGLYLIYSQVLFKGQGCPSTHVLL
THTISRIAVSYQTKVNLLSAIKSPCQRETPEGAEAKPWYEPIYLGGVFQL
EKGDRLSAEINRPDYLDFAESGQVYFGIIAL

TNF-Alpha delVal1 delVal13 Nucleic Acid Sequence

(SEQ ID NO: 6)
atgagcactgaaagcatgatccgggacgtggagctggccgaggaggcgct
ccccaagaagacaggggggccccagggctccaggcggtgcttgttcctca
gcctcttctccttcctgatcgtggcaggcgccaccacgctcttctgcctg
ctgcactttggagtgatcggcccccagagggaagagttccccagggacct
ctctctaatcagccctctggcccaggcaagatcatcttctcgaaccccga
gtgacaagcctgcccatgttgtagcaaaccctcaagctgaggggcagctc
cagtggctgaaccgccgggccaatgccctcctggccaatggcgtggagct
gagagataaccagctggtggtgccatcagagggcctgtacctcatctact
cccaggtcctcttcaagggccaaggctgcccctccacccatgtgctcctc
acccacaccatcagccgcatcgccgtctcctaccagaccaaggtcaacct
cctctctgccatcaagagcccctgccagagggagaccccagagggggctg
aggccaagccctggtatgagcccatctatctgggaggggtcttccagctg
gagaagggtgaccgactcagcgctgagatcaatcggcccgactatctcga
ctttgccgagtctgggcaggtctactttgggatcattgccctgtcg

TNF-Alpha delAla −3 to Val 13 Nucleic Acid Sequence

(SEQ ID NO: 38)
TCGAGTCGAGATGAGCACTGAAAGCATGATCCGGGACGTGGAGCTGGCCG
AGGAGGCGCTCCCCAAGAAGACAGGGGGGCCCCAGGGCTCCAGGCGGTGC
TTGTTCCTCAGCCTCTTCTCCTTCCTGATCGTGGCAGGCGCCACCACGCT
CTTCTGCCTGCTGCACTTTGGAGTGATCGGCCCCCAGAGGGAAGAGTTCC
CCAGGGACCTCTCTCTAATCAGCCCTCTGCAGGCAGCCCATGTTGTAGCA
AACCCTCAAGCTGAGGGGCAGCTCCAGTGGCTGAACCGCCGGGCCAATGC
CCTCCTGGCCAATGGCGTGGAGCTGAGAGATAACCAGCTGGTGGTGCCAT
CAGAGGGCCTGTACCTCATCTACTCCCAGGTCCTCTTCAAGGGCCAAGGC
TGCCCCTCCACCCATGTGCTCCTCACCCACACCATCAGCCGCATCGCCGT
CTCCTACCAGACCAAGGTCAACCTCCTCTCTGCCATCAAGAGCCCCTGCC
AGAGGGAGACCCCAGAGGGGGCTGAGGCCAAGCCCTGGTATGAGCCCATC
TATCTGGGAGGGGTCTTCCAGCTGGAGAAGGGTGACCGACTCAGCGCTGA
GATCAATCGGCCCGACTATCTCgACTTTGCCGAGTCTGGGCAGGTCTACT
TTGGGATCATTGCCCTGTCGTCG

TNF-alpha del Ala −3 and del of Val 1 through to and including Val 13 amino acid sequence (del −3 and del of 1-13 (but not deletion of −2 and −1)):

(SEQ ID NO: 39)
MSTESMIRDVELAEEALPKKTGGPQGSRRCLFLSLFSFLIVAGATTLFCL
LHFGVIGPQREEFPRDLSLISPLQAAHVVANPQAEGQLQWLNRRANALLA
NGVELRDNQLVVPSEGLYLIYSQVLFKGQGCPSTHVLLTHTISRIAVSYQ
TKVNLLSAIKSPCQRETPEGAEAKPWYEPIYLGGVFQLEKGDRLSAEINR
PDYLDFAESGQVYFGIIAL

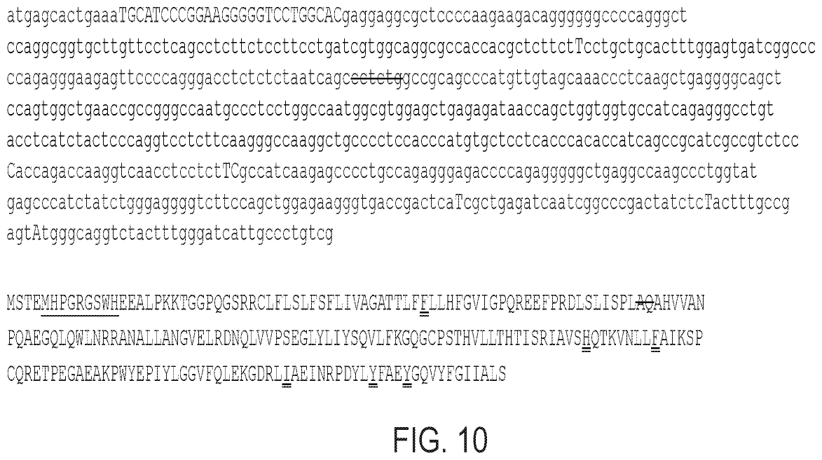

TNF-alpha mutant with del Ala-3 to Val13 nucleic acid sequence in addition to an example of a CIK motif mutation that prevents outside-in signaling and other mutations that interfere with TNFalpha binding to TNF Receptor 1 and TNF Receptor 2 (see FIG. 10)

(SEQ ID NO: 40)
ATGAGCACTGAAATGCATCCCGGAAGGGGGTCCTGGCACGAGGAGGCGCT
CCCCAAGAAGACAGGGGGGCCCCAGGGCTCCAGGCGGTGCTTGTTCCTCA
GCCTCTTCTCCTTCCTGATCGTGGCAGGCGCCACCACGCTCTTCTTCCTG
CTGCACTTTGGAGTGATCGGCCCCCAGAGGGAAGAGTTCCCCAGGGACCT
CTCTCTAATCAGCCCTCTGGCCCATGTTGTAGCAAACCCTCAAGCTGAGG
GGCAGCTCCAGTGGCTGAACCGCCGGGCCAATGCCCTCCTGGCCAATGGC
GTGGAGCTGAGAGATAACCAGCTGGTGGTGCCATCAGAGGGCCTGTACCT
CATCTACTCCCAGGTCCTCTTCAAGGGCCAAGGCTGCCCCTCCACCCATG
TGCTCCTCACCCACACCATCAGCCGCATCGCCGTCTCCCACCAGACCAAG
GTCAACCTCCTCTTCGCCATCAAGAGCCCCTGCCAGAGGGAGACCCCAGA
GGGGGCTGAGGCCAAGCCCTGGTATGAGCCCATCTATCTGGGAGGGGTCT
TCCAGCTGGAGAAGGGTGACCGACTCATCGCTGAGATCAATCGGCCCGAC
TATCTCTACTTTGCCGAGTATGGGCAGGTCTACTTTGGGATCATTGCCCT
GTCG

TNF-alpha mutant with del Ala-3 to Val13 amino acid sequence encoded by SEQ ID NO:40

(SEQ ID NO: 41)
MSTEMHPGRGSWHEEALPKKTGGPQGSRRCLFLSLFSFLIVAGATTLFFL
LHFGVIGPQREEFPRDLSLISPLAHVVANPQAEGQLQWLNRRANALLANG
VELRDNQLVVPSEGLYLIYSQVLFKGQGCPSTHVLLTHTISRIAVSHQTK
VNLLFAIKSPCQRETPEGAEAKPWYEPIYLGGVFQLEKGDRLIAEINRPD
YLYFAEYGQVYFGIIALS

In specific embodiments, a TNF-alpha mutant may comprise deletion of Ala-3 to Val13 but not also comprise a CIK motif mutation and a mutation that interferes with binding to TNF Receptor 1 and/or TNF Receptor 2.

For Reference, TNF Wild Type, 26 kD, Version Amino Acid Sequence

(SEQ ID NO: 7)
MSTESMIRDVELAEEALPKKTGGPQGSRRCLFLSLFSFLIVAGATTLFCL
LHFGVIGPQREEFPRDLSLISPLAQAVRSSSRTPSDKPVAHVVANPQAEG
QLQWLNRRANALLANGVELRDNQLVVPSEGLYLIYSQVLFKGQGCPSTHV
LLTHTISRIAVSYQTKVNLLSAIKSPCQRETPEGAEAKPWYEPIYLGGVF
QLEKGDRLSAEINRPDYLDFAESGQVYFGIIAL

For Reference, TNF Wild Type, 17 kD Version, Amino Acid Sequence

(SEQ ID NO: 8)
VRSSSRTPSDKPVAHVVANPQAEGQLQWLNRRANALLANGVELRDNQL
VVPSEGLYLIYSQVLFKGQGCPSTHVLLTHTISRIAVSYQTKVNLLSA
IKSPCQRETPEGAEAKPWYEPIYLGGVFQLEKGDRLSAEINRPDYLDF
AESGQVYFGIIAL

TNF-Alpha Mutants Lacking Intracellular TNF Signaling or TNF-Receptor Binding Capability

These mutants have mutations in the cytoplasmic signaling domain and/or in the TNF-receptor binding regions and therefore do not exert any biological activity as they lack reverse signaling capability and/or the ability to bind TNF-receptors, respectively. This allows for the TNF-alpha in the construct to be a target for TNF inhibitors, while exerting no biological activity.

In some embodiments of the disclosure, TNF-alpha mutants lack part or all of the intracytoplasmic domain of TNF-alpha such that the TNF-alpha mutant is unable to exert intracellular signaling (reverse signaling). The nonsecretable TNF-alpha mutants may or may not also be mutated to lack part or all of the intracytoplasmic domain.

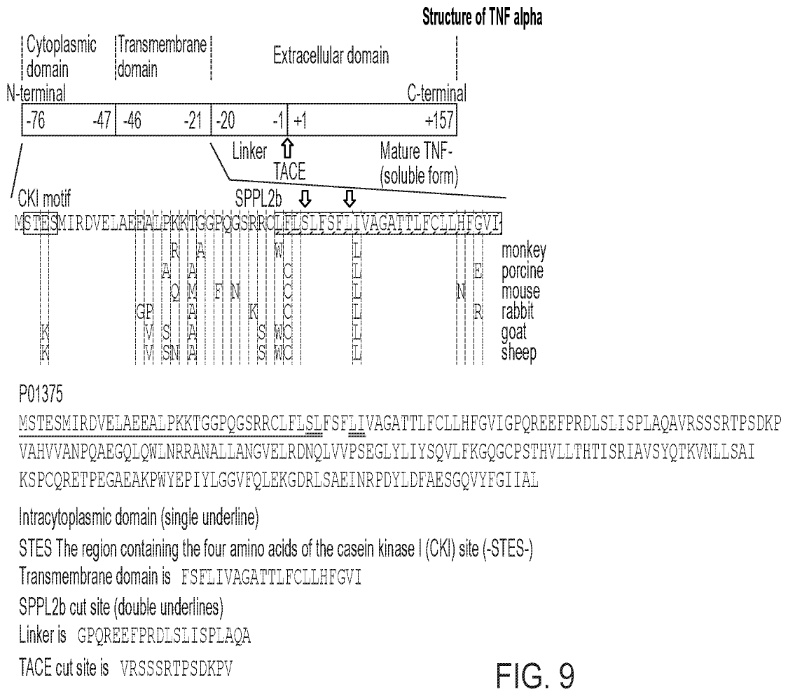

FIG. 9 provides some structure of TNF-alpha. As illustrated in FIG. 9, the intracytoplasmic domain comprises MSTESMIRDVELAEEALPKKTGGPQGSRRCLFL (SEQ ID NO:17). The casein kinase I (CKI) site is STES (SEQ ID NO:18). The transmembrane domain is FSFLIVAGATTLFCLLHFGVI (SEQ ID NO:19). The SPPL2b cut site is SL/LI. The linker comprises GPQREEFPRDLSLISPLAQA (SEQ ID NO:20). The TACE cute site is VRSSSRTPSDKPV (SEQ ID NO:21). P01375 refers to the UniProt number of the protein. The sequence in FIG. 9 refers to only part of the TNF protein.

Specific examples of TNF-alpha mutant for the CKI motif (mutated sequence underlined) for nucleic acid and amino acid, respectively, is as follows:

(SEQ ID NO: 22)
atgagcactgaaaTGCATCCCGGAAGGGGGTCCTGGCACgaggaggcg
ctccccaagaagacaggggggccccagggctccaggcggtgcttgttc
ctcagcctcttctccttcctgatcgtggcaggcgccaccacgctcttc
tgcctgctgcactttggagtgatcggcccccagagggaagagttcccc
agggacctctctctaatcagccctctggcccaggcagcccatgttgta
gcaaaccctcaagctgaggggcagctccagtggctgaaccgccgggcc
aatgccctcctggccaatggcgtggagctgagagataaccagctggtg
gtgccatcagagggcctgtacctcatctactcccaggtcctcttcaag
ggccaaggctgcccctccacccatgtgctcctcacccacaccatcagc
cgcatcgccgtctcctaccagaccaaggtcaacctcctctctgccatc
aagagcccctgccagagggagaccccagagggggctgaggccaagccc
tggtatgagcccatctatctgggaggggtcttccagctggagaagggt
gaccgactcagcgctgagatcaatcggcccgactatctcgactttgcc
gagtctgggcaggtctactttgggatcattgccctgcg
(SEQ ID NO: 23)
MSTEMHPGRGSWHEEALPKKTGGPQGSRRCLFLSLFSFLIVAGATTLF
CLLHFGVIGPQREEFPRDLSLISPLAQAVRSSSRTPSDKPVAHVVANP
QAEGQLQWLNRRANALLANGVELRDNQLVVPSEGLYLIYSQVLFKGQG
CPSTHVLLTHTISRIAVSYQTKVNLLSAIKSPCQRETPEGAEAKPWYE
PIYLGGVFQLEKGDRLSAEINRPDYLDFAESGQVYFGIIAL

One example of a TNF-alpha mutant having a mutation at M-71K in the intracytoplasmic sequence and another mutation at Y87H (mutated sequences underlined) for nucleic acid and amino acid, respectively, is as follows:

(SEQ ID NO: 24)
atgagcactgaaagcaAgatccgggacgtggagctggccgaggaggcg
ctccccaagaagacaggggggccccagggctccaggcggtgcttgttc
ctcagcctcttctccttcctgatcgtggcaggcgccaccacgctcttc
tgcctgctgcactttggagtgatcggcccccagagggaagagttcccc
agggacctctctctaatcagccctctggcccaggcagcccatgttgta
gcaaaccctcaagctgaggggcagctccagtggctgaaccgccgggcc
aatgccctcctggccaatggcgtggagctgagagataaccagctggtg
gtgccatcagagggcctgtacctcatctactcccaggtcctcttcaag
ggccaaggctgcccctccacccatgtgctcctcacccacaccatcagc
cgcatcgccgtctccCaccagaccaaggtcaacctcctctctgccatc
aagagcccctgccagagggagaccccagagggggctgaggccaagccc
tggtatgagcccatctatctgggaggggtcttccagctggagaagggt
gaccgactcagcgctgagatcaatcggcccgactatctcgactttgcc
gagtctgggcaggtctactttgggatcattgccctgtcg
(SEQ ID NO: 25)
MSTESKIRDVELAEEALPKKTGGPQGSRRCLFLSLFSFLIVAGATTLF
CLLHFGVIGPQREEFPRDLSLISPLAQAAHVVANPQAEGQLQWLNRRA
NALLANGVELRDNQLVVPSEGLYLIYSQVLFKGQGCPSTHVLLTHTIS
RIAVSHQTKVNLLSAIKSPCQRETPEGAEAKPWYEPIYLGGVFQLEKG
DRLSAEINRPDYLDFAESGQVYFGIIAL

One example of a TNF-alpha mutant having a mutation at S95F and C-28F (mutated sequences underlined) for nucleic acid and amino acid, respectively, is as follows:

(SEQ ID NO: 26)
atgagcactgaaagcatgatccgggacgtggagctggccgaggaggcg
ctccccaagaagacaggggggccccagggctccaggcggtgcttgttc
ctcagcctcttctccttcctgatcgtggcaggcgccaccacgctcttc
tTcctgctgcactttggagtgatcggcccccagagggaagagttcccc
agggacctctctctaatcagccctctggcccaggcagcccatgttgta
gcaaaccctcaagctgaggggcagctccagtggctgaaccgccgggcc
aatgccctcctggccaatggcgtggagctgagagataaccagctggtg
gtgccatcagagggcctgtacctcatctactcccaggtcctcttcaag
ggccaaggctgcccctccacccatgtgctcctcacccacaccatcagc
cgcatcgccgtctcctaccagaccaaggtcaacctcctctTCgccatc
aagagcccctgccagagggagaccccagagggggctgaggccaagccc
tggtatgagcccatctatctgggaggggtcttccagctggagaagggt
gaccgactcagcgctgagatcaatcggcccgactatctcgactttgcc
gagtctgggcaggtctactttgggatcattgccctgtcg
(SEQ ID NO: 27)
MSTESMIRDVELAEEALPKKTGGPQGSRRCLFLSLFSFLIVAGATTLF
FLLHFGVIGPQREEFPRDLSLISPLAQAAHVVANPQAEGQLQWLNRRA
NALLANGVELRDNQLVVPSEGLYLIYSQVLFKGQGCPSTHVLLTHTIS
RIAVSYQTKVNLLFAIKSPCQRETPEGAEAKPWYEPIYLGGVFQLEKG
DRLSAEINRPDYLDFAESGQVYFGIIAL

One example of a TNF-alpha mutant having a mutation at S133I and S147Y (mutated sequences underlined) for nucleic acid and amino acid, respectively, is as follows:

(SEQ ID NO: 28)
atgagcactgaaagcatgatccgggacgtggagctggccgaggaggcg
ctccccaagaagacaggggggccccagggctccaggcggtgcttgttc
ctcagcctcttctccttcctgatcgtggcaggcgccaccacgctcttc
tgcctgctgcactttggagtgatcggcccccagagggaagagttcccc
agggacctctctctaatcagccctctggcccaggcagcccatgttgta
gcaaaccctcaagctgaggggcagctccagtggctgaaccgccgggcc
aatgccctcctggccaatggcgtggagctgagagataaccagctggtg
gtgccatcagagggcctgtacctcatctactcccaggtcctcttcaag
ggccaaggctgcccctccacccatgtgctcctcacccacaccatcagc
cgcatcgccgtctcctaccagaccaaggtcaacctcctctctgccatc
aagagcccctgccagagggagaccccagagggggctgaggccaagccc
tggtatgagcccatctatctgggaggggtcttccagctggagaagggt
gaccgactcaTcgctgagatcaatcggcccgactatctcgactttgcc
gagtAtgggcaggtctactttgggatcattgccctgtcg
(SEQ ID NO: 29)
MSTESMIRDVELAEEALPKKTGGPQGSRRCLFLSLFSFLIVAGATTLF
CLLHFGVIGPQREEFPRDLSLISPLAQAAHVVANPQAEGQLQWLNRRA
NALLANGVELRDNQLVVPSEGLYLIYSQVLFKGQGCPSTHVLLTHTIS
RIAVSYQTKVNLLSAIKSPCQRETPEGAEAKPWYEPIYLGGVFQLEKG
DRLIAEINRPDYLDFAEYGQVYFGIIAL

One example of a TNF-alpha mutant having a mutation at Asp143Tyr and a deletion of Ala at position −1 (mutated sequence underlined and deleted sequence shown by strikethrough) for nucleic acid and amino acid, respectively, is as follows:

(SEQ ID NO: 30)
atgagcactgaaagcatgatccgggacgtggagctggccgaggaggcg
ctccccaagaagacaggggggccccagggctccaggcggtgcttgttc
ctcagcctcttctccttcctgatcgtggcaggcgccaccacgctcttc
tgcctgctgcactttggagtgatcggccoccagagggaagagttcccc
agggacctctctctaatcagccctctgcaggcagcccatgttgtagca
aaccctcaagctgaggggcagctccagtggctgaaccgccgggccaat
gccctcctggccaatggcgtggagctgagagataaccagctggtggtg
ccatcagagggcctgtacctcatctactcccaggtcctcttcaagggc
caaggctgcccctccacccatgtgctcctcacccacaccatcagccgc
atcgccgtctcctaccagaccaaggtcaacctcctctctgccatcaag
agcccctgccagagggagaccccagagggggctgaggccaagccctgg
tatgagcccatctatctgggaggggtcttccagctggagaagggtgac
cgactcagcgctgagatcaatcggcccgactatctcTactttgccgag
tctgggcaggtctactttgggatcattgccctgtcg
(SEQ ID NO: 31)
MSTESMIRDVELAEEALPKKTGGPQGSRRCLFLSLFSFLIVAGATTLF
CLLHFGVIGPQREEFPRDLSLISPLAQAHVVANPQAEGQLQWLNRRAN
ALLANGVELRDNQLVVPSEGLYLIYSQVLFKGQGCPSTHVLLTHTISR
IAVSYQTKVNLLSAIKSPCQRETPEGAEAKPWYEPIYLGGVFQLEKGD
RLSAEINRPDYLYFAESGQVYFGIIAL

Versions of SEQ ID NO:30 and SEQ ID NO:31 that lack the deleted sequences are as follows, respectively (with the mutated sequence still underlined).

(SEQ ID NO: 32)
atgagcactgaaagcatgatccgggacgtggagctggccgaggaggcg
ctccccaagaagacaggggggccccagggctccaggcggtgcttgttc
ctcagcctcttctccttcctgatcgtggcaggcgccaccacgctcttc
tgcctgctgcactttggagtgatcggcccccagagggaagagttcccc
agggacctctctctaatcagccctctgcaggcagcccatgttgtagca
aaccctcaagctgaggggcagctccagtggctgaaccgccgggccaat
gccctcctggccaatggcgtggagctgagagataaccagctggtggtg
ccatcagagggcctgtacctcatctactcccaggtcctcttcaagggc
caaggctgcccctccacccatgtgctcctcacccacaccatcagccgc
atcgccgtctcctaccagaccaaggtcaacctcctctctgccatcaag
agcccctgccagagggagaccccagagggggctgaggccaagccctgg
tatgagcccatctatctgggaggggtcttccagctggagaagggtgac
cgactcagcgctgagatcaatcggcccgactatctcTactttgccgag
tctgggcaggtctactttgggatcattgccctgtcg
(SEQ ID NO: 33)
MSTESMIRDVELAEEALPKKTGGPQGSRRCLFLSLFSFLIVAGATTLF
CLLHFGVIGPQREEFPRDLSLISPLAQAHVVANPQAEGQLQWLNRRAN
ALLANGVELRDNQLVVPSEGLYLIYSQVLFKGQGCPSTHVLLTHTISR
IAVSYQTKVNLLSAIKSPCQRETPEGAEAKPWYEPIYLGGVFQLEKGD
RLSAEINRPDYLYFAESGQVYFGIIAL

One example of a TNF-alpha mutant having a combination of the CIK motif mutation and the above-referenced mutations are as follows, with the mutations underlined:

(SEQ ID NO: 34)
ATGCTCGAGtcgagatgagcactgaaaTGCATCCCGGAAGGGGGTCCT
GGCACgaggaggcgctccccaagaagacaggggggccccagggctcca
ggcggtgcttgttcctcagcctcttctccttcctgatcgtggcaggcg
ccaccacgctcttctTcctgctgcactttggagtgatcggcccccaga
gggaagagttccccagggacctctctctaatcagccctctggcagccc
atgttgtagcaaaccctcaagctgaggggcagctccagtggctgaacc
gccgggccaatgccctcctggccaatggcgtggagctgagagataacc
agctggtggtgccatcagagggcctgtacctcatctactcccaggtcc
tcttcaagggccaaggctgcccctccacccatgtgctcctcacccaca
ccatcagccgcatcgccgtctccCaccagaccaaggtcaacctcctct
TCgccatcaagagcccctgccagagggagaccccagagggggctgagg
ccaagccctggtatgagcccatctatctgggaggggtcttccagctgg
agaagggtgaccgactcaTcgctgagatcaatcggcccgactatctcT
actttgccgagtAtgggcaggtctactttgggatcattgccctgtcg
(SEQ ID NO: 35)
MSTEMHPGRGSWHEEALPKKTGGPQGSRRCLFLSLFSFLIVAGATTLF
FLLHFGVIGPQREEFPRDLSLISPLAQAHVVANPQAEGQLQWLNRRAN
ALLANGVELRDNQLVVPSEGLYLIYSQVLFKGQGCPSTHVLLTHTISR
IAVSHQTKVNLLFAIKSPCQRETPEGAEAKPWYEPIYLGGVFQLEKGD
RLIAEINRPDYLYFAEYGQVYFGIIAL

### III. Therapeutic Gene(s)

In some cases, cells expressing the TNF-alpha mutant(s) may also express one or more therapeutic genes. In cases where more than one therapeutic gene is employed, the therapeutic genes may or may not be of the same type of molecule. For example, in addition to the TNF-alpha mutant, a single cell may also express an engineered receptor, a cytokine, cytokine receptor, homing receptor, chemokine receptor, or a combination thereof. Encompassed herein are therapeutic gene nucleic acids; therapeutic gene products, including polypeptides; vectors comprising the therapeutic gene nucleic acid; and cells harboring any thereof.

In particular embodiments, the mutant is co-expressed with at least one therapeutic gene, including a therapeutic transgene. The therapeutic transgene may be of any kind, but in specific embodiments it encodes an engineered receptor. Examples of engineered receptors include at least a T-cell receptor, chimeric antigen receptor (CAR), chemokine receptor, cytokine receptor, homing receptor, or a combination thereof. Any engineered receptor may target any particular ligand, such as an antigen, including a cancer antigen (such as a tumor antigen). The cancer antigens may be of any kind, including those associated with a particular cancer to be treated and that is desired to be targeted for specific elimination of the cancer.

In cases wherein the therapeutic gene product is an engineered receptor, the receptor comprises an antigen binding domain that may target any antigen, such as a tumor antigen. The antigen binding domain may comprise an scFv, for example. Antigenic molecules may come from infectious agents, auto-/self-antigens, tumor-/cancer-associated antigens, or tumor neoantigens, for example. Examples of antigens that may be targeted include but are not limited to antigens expressed on B-cells; antigens expressed on carcinomas, sarcomas, lymphomas, leukemia, germ cell tumors, and blastomas; antigens expressed on various immune cells; and antigens expressed on cells associated with various hematologic diseases, autoimmune diseases, and/or inflammatory diseases. Examples of specific antigens to target include CD19, CD5, CD99, CD33, CLL1, CD123, 4-1BB, 5T4, adenocarcinoma antigen, alpha-fetoprotein, BAFF, B-lymphoma cell, C242 antigen, CA-125, carbonic anhydrase 9 (CA-IX), C-MET, CCR4, CD152, CD20, CD200, CD22, CD221, CD23 (IgE receptor), CD28, CD30 (TNFRSF8), CD33, CD4, CD40, CD44 v6, CD51, CD52, CD56, CD74, CD80, CEA, CNT0888, CTLA-4, DRS, EGFR, EpCAM, CD3, FAP, fibronectin extra domain-B, folate receptor 1, GD2, GD3 ganglioside, glycoprotein 75, GPNMB, HER2/neu, HGF, human scatter factor receptor kinase, IGF-1 receptor, IGF-I, IgG1, L1-CAM, IL-13, IL-6, insulin-like growth factor I receptor, integrin-α5β1, integrinαvβ3, MORAb-009, MS4A1, MUC1, mucin CanAg, N-glycolylneuraminic acid, NPC-1C, PDGF-R .alpha., PDL192, phosphatidylserine, prostatic carcinoma cells, RANKL, RON, ROR1, SCH 900105, SDC1, SLAMF7, TAG-72, tenascin C, TGF β2, TGF-β, TRAIL-R1, TRAIL-R2, tumor antigen CTAA16.88, VEGF-A, VEGFR-1, VEGFR2, vimentin, and combinations thereof. Any antigen receptor that may be utilized in methods and compositions of the disclosure may target any one of the above-referenced antigens, or one or more others, and such an antigen receptor may be a CAR or a TCR. The same cells for therapy may utilize both a CAR and a TCR, in specific embodiments.

In cases wherein the therapeutic gene encodes a CAR, the CAR may be first generation, second generation, or third or subsequent generation, for example. The CAR may or may not be bispecific to two or more different antigens. The CAR may comprise one or more co-stimulatory domains. Each co-stimulatory domain may comprise the costimulatory domain of any one or more of, for example, members of the TNFR superfamily, CD28, CD137 (4-1BB), CD134 (OX40), Dap10, DAP12, CD27, CD2, CD5, ICAM-1, LFA-1 (CD11a/CD18), Lck, TNFR-I, TNFR-II, Fas, CD30, CD40 or combinations thereof, for example. In specific embodiments, the CAR comprises CD3zeta. In certain embodiments, the CAR lacks one or more specific costimulatory domains; for example, the CAR may lack 4-1BB.

In a specific embodiment, the CAR comprises at least DAP12 as a costimulatory domain, and in certain aspects the CAR polypeptide comprises a particular DAP12 amino acid sequence or is encoded by a particular DAP12 nucleic acid sequence. Examples are as follows:

DAP12 amino acid sequence
(SEQ ID NO: 9)
MGGLEPCSRLLLLPLLLAVSGLRPVQAQAQSDCSCSTVSPGVLAGIVM
GDLVLTVLIALAVYFLGRLVPRGRGAAEAATRKQRITETESPYQELQG
QRSDVYSDLNTQRPYYK
DAP12 nucleic acid sequence
(SEQ ID NO: 10)
ATGGGGGGACTTGAACCCTGCAGCAGGCTCCTGCTCCTGCCTCTCCTG
CTGGCTGTAAGTGGTCTCCGTCCTGTCCAGGCCCAGGCCCAGAGCGAT
TGCAGTTGCTCTACGGTGAGCCCGGGCGTGCTGGCAGGGATCGTGATG
GGAGACCTGGTGCTGACAGTGCTCATTGCCCTGGCCGTGTACTTCCTG
GGCCGGCTGGTCCCTCGGGGGCGAGGGGCTGCGGAGGCAGCGACCCGG
AAACAGCGTATCACTGAGACCGAGTCGCCTTATCAGGAGCTCCAGGGT
CAGAGGTCGGATGTCTACAGCGACCTCAACACACAGAGGCCGTATTAC
AAATGA

In a specific embodiment, the CAR comprises at least CD28 as a costimulatory domain, and in certain aspects the CAR polypeptide comprises a particular CD28 amino acid sequence or is encoded by a particular CD28 nucleic acid sequence. Examples are as follows:

CD28 amino acid sequence
(SEQ ID NO: 11)
KFWVLVVVGGVLACYSLLVTVAFIIFWVRSKRSRLLHSDYMNMTPRRP
GPTRKHYQPYAPPRDFAAYRSRVKFSRSADAPAYQQGQNQLYNELNLG
RREEYDVLDKRRGRDPEMGGKPRRKNPQEGLYNELQKDKMAEAYSEIG
MKGERRRGKGHDGLYQGLSTATKDTYDALHMQALPPRG
CD28 nucleic acid sequence
(SEQ ID NO: 12)
ATTTTGGGTGCTGGTGGTGGTTGGTGGAGTCCTGGCTTGCTATAGCTT
GCTAGTAACAGTGGCCTTTATTATTTTCTGGGTGAGGAGTAAGAGGAG
CAGGCTCCTGCACAGTGACTACATGAACATGACTCCCCGCCGCCCCGG
GCCCACCCGCAAGCATTACCAGCCCTATGCCCCACCACGCGACTTCGC
AGCCTATCGCTCACGCG

In particular embodiments, the CAR polypeptide comprises an extracellular spacer domain that links the antigen binding domain and the transmembrane domain. Extracellular spacer domains may include, but are not limited to, Fc fragments of antibodies or fragments or derivatives thereof, hinge regions of antibodies or fragments or derivatives thereof, CH2 regions of antibodies, CH3 regions antibodies, artificial spacer sequences or combinations thereof. Examples of extracellular spacer domains include but are not limited to CD8-alpha hinge, artificial spacers made of polypeptides such as Gly3, or CH1, CH3 domains of IgGs (such as human IgG1 or IgG4). In specific cases, the extracellular spacer domain may comprise (i) a hinge, CH2 and CH3 regions of IgG4, (ii) a hinge region of IgG4, (iii) a hinge and CH2 of IgG4, (iv) a hinge region of CD8-alpha, (v) a hinge, CH2 and CH3 regions of IgG1, (vi) a hinge region of IgG1 or (vi) a hinge and CH2 of IgG1 or a combination thereof.

In specific embodiments, the hinge is from IgG1 and in certain aspects the CAR polypeptide comprises a particular IgG1 hinge amino acid sequence or is encoded by a particular IgG1 hinge nucleic acid sequence. Examples are as follows:

IgG1 hinge amino acid sequence
(SEQ ID NO: 13)
SYVTVSSQDPAEPKSPDKTHTCPPCPAPELLGGPSVFLFPPKPKDTLM
ISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQV
YTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLS
PGKKDPK
IgG1 hinge nucleic acid sequence
(SEQ ID NO: 14)
GTACGTCACTGTCTCTTCACAGGATCCCGCCGAGCCCAAATCTCCTGA
CAAAACTCACACATGCCCACCGTGCCCAGCACCTGAACTCCTGGGGGG
ACCGTCAGTCTTCCTCTTCCCCCCAAAACCCAAGGACACCCTCATGAT
CTCCCGGACCCCTGAGGTCACATGCGTGGTGGTGGACGTGAGCCACGA
AGACCCTGAGGTCAAGTTCAACTGGTACGTGGACGGCGTGGAGGTGCA
TAATGCCAAGACAAAGCCGCGGGAGGAGCAGTACAACAGCACGTACCG
TGTGGTCAGCGTCCTCACCGTCCTGCACCAGGACTGGCTGAATGGCAA
GGAGTACAAGTGCAAGGTCTCCAACAAAGCCCTCCCAGCCCCCATCGA
GAAAACCATCTCCAAAGCCAAAGGGCAGCCCCGAGAACCACAGGTGTA
CACCCTGCCCCCATCCCGGGATGAGCTGACCAAGAACCAGGTCAGCCT
GACCTGCCTGGTCAAAGGCTTCTATCCCAGCGACATCGCCGTGGAGTG
GGAGAGCAATGGGCAACCGGAGAACAACTACAAGACCACGCCTCCCGT
GCTGGACTCCGACGGCTCCTTCTTCCTCTACAGCAAGCTCACCGTGGA
CAAGAGCAGGTGGCAGCAGGGGAACGTCTTCTCATGCTCCGTGATGCA
TGAGGCTCTGCACAACCACTACACGCAGAAGAGCCTCTCCCTGTCTCC
GGGTAAAAAAGATC

### IV. Vectors

The TNF-alpha mutant(s) may be delivered to the recipient cell by any suitable vector, including by a viral vector or by a non-viral vector. Examples of viral vectors include at least retroviral, lentiviral, adenoviral, or adeno-associated viral vectors. Examples of non-viral vectors include at least plasmids, transposons, lipids, nanoparticles, and so forth.

In cases wherein the cell is transduced with a vector encoding the TNF-alpha mutant and also requires transduction of another gene into the cell, such as a therapeutic gene product, the TNF-alpha mutant gene and therapeutic gene may or may not be comprised on or with the same vector. In some cases, the TNF-alpha mutant gene and the therapeutic gene are expressed from the same vector molecule, such as the same viral vector molecule. In such cases, the expression of the TNF-alpha mutant gene and the therapeutic gene may or may not be regulated by the same regulatory element(s). When the TNF-alpha mutant gene and the therapeutic gene are on the same vector, they may or may not be expressed as separate polypeptides. In cases wherein they are expressed as separate polypeptides, they may be separated on the vector by a 2A element or IRES element, for example. In some embodiments the TNF-alpha mutant and the therapeutic gene product are produced as a fusion protein.

In particular embodiments, the TNF-alpha mutant gene is expressed from a multicistronic vector. The multicistronic vector may encode at least one therapeutic gene in addition to the TNF-alpha mutant gene. In specific embodiments, the multicistronic vector encodes the TNF-alpha mutant and at least one engineered receptor, such as a T-cell receptor and/or a CAR. In some cases, the multicistronic vector encodes at least one TNF-alpha mutant, at least one engineered receptor, and at least one cytokine. The cytokine may be of a particular type of cytokine, such as human or mouse or any species. In specific cases, the cytokine is interleukin (IL)15, IL12, IL2, IL18, and/or IL21.

One example of nucleic acid sequence for a vector that encodes a TNF-alpha mutant del Val1 del Pro12 and that separately encodes a CD19-specific CAR with an IgG1 hinge, CD28, and CD3zeta and that separately encodes IL15 is as follows:

(SEQ ID NO: 15)
AATGAAAGACCCCACCTGTAGGTTTGGCAAGCTAGCTTAAGTAACGCCATTTTGCAAGGCATGGAAAAATA
CATAACTGAGAATAGAAAAGTTCAGATCAAGGTCAGGAACAGATGGAACAGCTGAATATGGGCCAAACAGGATATCT
GTGGTAAGCAGTTCCTGCCCCGGCTCAGGGCCAAGAACAGATGGAACAGCTGAATATGGGCCAAACAGGATATCTGT
GGTAAGCAGTTCCTGCCCCGGCTCAGGGCCAAGAACAGATGGTCCCCAGATGCGGTCCAGCCCTCAGCAGTTTCTAG
AGAACCATCAGATGTTTCCAGGGTGCCCCAAGGACCTGAAATGACCCTGTGCCTTATTTGAACTAACCAATCAGTTC
GCTTCTCGCTTCTGTTCGCGCGCTTATGCTCCCCGAGCTCAATAAAAGAGCCCACAACCCCTCACTCGGGGCGCCAG
TCCTCCGATTGACTGAGTCGCCCGGGTACCCGTGTATCCAATAAACCCTCTTGCAGTTGCATCCGACTTGTGGTCTC
GCTGTTCCTTGGGAGGGTCTCCTCTGAGTGATTGACTACCCGTCAGCGGGGGTCTTTCATTTGGGGGCTCGTCCGGG
ATCGGGAGACCCCTGCCCAGGGACCACCGACCCACCACCGGGAGGTAAGCTGGCCAGCAACTTATCTGTGTCTGTCC
GATTGTCTAGTGTCTATGACTGATTTTATGCGCCTGCGTCGGTACTAGTTAGCTAACTAGCTCTGTATCTGGCGGAC
CCGTGGTGGAACTGACGAGTTCGGAACACCCGGCCGCAACCCTGGGAGACGTCCCAGGGACTTCGGGGGCCGTTTTT
GTGGCCCGACCTGAGTCCTAAAATCCCGATCGTTTAGGACTCTTTGGTGCACCCCCCTTAGAGGAGGGATATGTGGT
TCTGGTAGGAGACGAGAACCTAAAACAGTTCCCGCCTCCGTCTGAATTTTTGCTTTCGGTTTGGGACCGAAGCCGCG
CCGCGCGTCTTGTCTGCTGCAGCATCGTTCTGTGTTGTCTCTGTCTGACTGTGTTTCTGTATTTGTCTGAAAATATG
GGCCCGGGCTAGCCTGTTACCACTCCCTTAAGTTTGACCTTAGGTCACTGGAAAGATGTCGAGCGGATCGCTCACAA
CCAGTCGGTAGATGTCAAGAAGAGACGTTGGGTTACCTTCTGCTCTGCAGAATGGCCAACCTTTAACGTCGGATGGC
CGCGAGACGGCACCTTTAACCGAGACCTCATCACCCAGGTTAAGATCAAGGTCTTTTCACCTGGCCCGCATGGACAC
CCAGACCAGGTGGGGTACATCGTGACCTGGGAAGCCTTGGCTTTTGACCCCCCTCCCTGGGTCAAGCCCTTTGTACA
CCCTAAGCCTCCGCCTCCTCTTCCTCCATCCGCCCCGTCTCTCCCCCTTGAACCTCCTCGTTCGACCCCGCCTCGAT
CCTCCCTTTATCCAGCCCTCACTCCTTCTCTAGGCGCCCCCATATGGCCATATGAGATCTTATATGGGGCACCCCCG
CCCCTTGTAAACTTCCCTGACCCTGACATGACAAGAGTTACTAACAGCCCCTCTCTCCAAGCTCACTTACAGGCTCT
CTACTTAGTCCAGCACGAAGTCTGGAGACCTCTGGCGGCAGCCTACCAAGAACAACTGGACCGACCGGTGGTACCTC
ACCCTTACCGAGTCGGCGACACAGTGTGGGTCCGCCGACACCAGACTAAGAACCTAGAACCTCGCTGGAAAGGACCT
TACACAGTCCTGCTGACCACCCCCACCGCCCTCAAAGTAGACGGCATCGCAGCTTGGATACACGCCGCCCACGTGAA
GGCTGCCGACCCCGGGGGTGGACCATCCTCTAGACTGCCATGCTCGAGATGAGCACTGAAAGCATGATCCGGGACGT
GGAGCTGGCCGAGGAGGCGCTCCCCAAGAAGACAGGGGGGCCCCAGGGCTCCAGGCGGTGCTTGTTCCTCAGCCTCT
TCTCCTTCCTGATCGTGGCAGGCGCCACCACGCTCTTCTGCCTGCTGCACTTTGGAGTGATCGGCCCCCAGAGGGAA
GAGTTCCCCAGGGACCTCTCTCTAATCAGCCCTCTGGCCCAGGCAAGATCATCTTCTCGAACCCCGAGTGACAAGGT
AGCCCATGTTGTAGCAAACCCTCAAGCTGAGGGGCAGCTCCAGTGGCTGAACCGCCGGGCCAATGCCCTCCTGGCCA
ATGGCGTGGAGCTGAGAGATAACCAGCTGGTGGTGCCATCAGAGGGCCTGTACCTCATCTACTCCCAGGTCCTCTTC
AAGGGCCAAGGCTGCCCCTCCACCCATGTGCTCCTCACCCACACCATCAGCCGCATCGCCGTCTCCTACCAGACCAA
GGTCAACCTCCTCTCTGCCATCAAGAGCCCCTGCCAGAGGGAGACCCCAGAGGGGGCTGAGGCCAAGCCCTGGTATG
AGCCCATCTATCTGGGAGGGGTCTTCCAGCTGGAGAAGGGTGACCGACTCAGCGCTGAGATCAATCGGCCCGACTAT
CTCGACTTTGCCGAGTCTGGGCAGGTCTACTTTGGGATCATTGCCCTGTCGCGAGCCGAGGGCAGGGGAAGTCTTCT
AACATGCGGGGACGTGGAGGAAAATCCCGGGCCCATGGAGTTTGGGCTGAGCTGGCTTTTTCTTGTGGCTATTTTAA
AAGGTGTCCAGTGCTCTAGAGACATCCAGATGACACAGACTACATCCTCCCTGTCTGCCTCTCTGGGAGACAGAGTC
ACCATCAGTTGCAGGGCAAGTCAGGACATTAGTAAATATTTAAATTGGTATCAGCAGAAACCAGATGGAACTGTTAA
ACTCCTGATCTACCATACATCAAGATTACACTCAGGAGTCCCATCAAGGTTCAGTGGCAGTGGGTCTGGAACAGATT
ATTCTCTCACCATTAGCAACCTGGAGCAAGAAGATATTGCCACTTACTTTTGCCAACAGGGTAATACGCTTCCGTAC
ACGTTCGGAGGGGGGACCAAGCTGGAGCTGAAACGTGGTGGTGGTGGTTCTGGTGGTGGTGGTTCTGGCGGCGGCGG
CTCCGGTGGTGGTGGATCCGAGGTGCAGCTGCAGCAGTCTGGACCTGGCCTGGTGGCGCCCTCACAGAGCCTGTCCG
TCACATGCACTGTCTCAGGGGTCTCATTACCCGACTATGGTGTAAGCTGGATTCGCCAGCCTCCACGAAAGGGTCTG
GAGTGGCTGGGAGTAATATGGGGTAGTGAAACCACATACTATAATTCAGCTCTCAAATCCAGACTGACCATCATCAA
GGACAACTCCAAGAGCCAAGTTTTCTTAAAAATGAACAGTCTGCAAACTGATGACACAGCCATTTACTACTGTGCCA
AACATTATTACTACGGTGGTAGCTATGCTATGGACTACTGGGGCCAAGGGACCACGGTCACTGTCTCCTCGTACGTC
ACTGTCTCTTCACAGGATCCCGCCGAGCCCAAATCTCCTGACAAAACTCACACATGCCCACCGTGCCCAGCACCTGA
ACTCCTGGGGGGACCGTCAGTCTTCCTCTTCCCCCCAAAACCCAAGGACACCCTCATGATCTCCCGGACCCCTGAGG
TCACATGCGTGGTGGTGGACGTGAGCCACGAAGACCCTGAGGTCAAGTTCAACTGGTACGTGGACGGCGTGGAGGTG
CATAATGCCAAGACAAAGCCGCGGGAGGAGCAGTACAACAGCACGTACCGTGTGGTCAGCGTCCTCACCGTCCTGCA
CCAGGACTGGCTGAATGGCAAGGAGTACAAGTGCAAGGTCTCCAACAAAGCCCTCCCAGCCCCCATCGAGAAAACCA
TCTCCAAAGCCAAAGGGCAGCCCCGAGAACCACAGGTGTACACCCTGCCCCCATCCCGGGATGAGCTGACCAAGAAC
CAGGTCAGCCTGACCTGCCTGGTCAAAGGCTTCTATCCCAGCGACATCGCCGTGGAGTGGGAGAGCAATGGGCAACC
GGAGAACAACTACAAGACCACGCCTCCCGTGCTGGACTCCGACGGCTCCTTCTTCCTCTACAGCAAGCTCACCGTGG
ACAAGAGCAGGTGGCAGCAGGGGAACGTCTTCTCATGCTCCGTGATGCATGAGGCTCTGCACAACCACTACACGCAG
AAGAGCCTCTCCCTGTCTCCGGGTAAAAAAGATCCCAAATTTTGGGTGCTGGTGGTGGTTGGTGGAGTCCTGGCTTG
CTATAGCTTGCTAGTAACAGTGGCCTTTATTATTTTCTGGGTGAGGAGTAAGAGGAGCAGGCTCCTGCACAGTGACT
ACATGAACATGACTCCCCGCCGCCCCGGGCCCACCCGCAAGCATTACCAGCCCTATGCCCCACCACGCGACTTCGCA
GCCTATCGCTCACGCGTGAAGTTCAGCAGGAGCGCAGACGCCCCCGCGTACCAGCAGGGCCAGAACCAGCTCTATAA
CGAGCTCAATCTAGGACGAAGAGAGGAGTACGATGTTTTGGACAAAAGACGTGGCCGGGACCCTGAGATGGGGGGAA
AGCCGAGAAGGAAGAACCCTCAGGAAGGCCTGTACAATGAACTGCAGAAAGATAAGATGGCGGAGGCCTACAGTGAG
ATTGGGATGAAAGGCGAGCGCCGGAGGGGCAAGGGGCACGATGGCCTTTACCAGGGTCTCAGTACAGCCACCAAGGA
CACCTACGACGCCCTTCACATGCAGGCCCTGCCCCCTCGCGGACCGCAGTGTACTAATTATGCTCTCTTGAAATTGG
CTGGAGATGTTGAGAGCAATCCCGGGCCCATGCGCATTAGCAAGCCCCACCTGCGGAGCATCAGCATCCAGTGCTAC
CTGTGCCTGCTGCTGAACAGCCACTTCCTGACCGAGGCCGGCATCCACGTGTTCATCCTGGGCTGCTTCAGCGCCGG
ACTGCCCAAGACCGAGGCCAACTGGGTGAACGTGATCAGCGACCTGAAGAAGATCGAGGACCTGATCCAGAGCATGC
ACATCGACGCCACCCTGTACACCGAGAGCGACGTGCACCCCAGCTGCAAGGTGACCGCCATGAAGTGCTTTCTGCTG
GAACTGCAGGTGATCAGCCTGGAAAGCGGCGACGCCAGCATCCACGACACCGTGGAGAACCTGATCATCCTGGCCAA
CAACAGCCTGAGCAGCAACGGCAACGTGACCGAGAGCGGCTGCAAAGAGTGCGAGGAACTGGAAGAGAAGAACATCA
AAGAGTTTCTGCAGAGCTTCGTGCACATCGTGCAGATGTTCATCAACACCAGCTGACAATTGCGCGTCATCATCGAT
CCGGATTAGTCCAATTTGTTAAAGACAGGATATCAGTGGTCCAGGCTCTAGTTTTGACTCAACAATATCACCAGCTG
AAGCCTATAGAGTACGAGCCATAGATAAAATAAAAGATTTTATTTAGTCTCCAGAAAAAGGGGGGAATGAAAGACCC
CACCTGTAGGTTTGGCAAGCTAGCTTAAGTAACGCCATTTTGCAAGGCATGGAAAAATACATAACTGAGAATAGAGA
AGTTCAGATCAAGGTCAGGAACAGATGGAACAGCTGAATATGGGCCAAACAGGATATCTGTGGTAAGCAGTTCCTGC
CCCGGCTCAGGGCCAAGAACAGATGGAACAGCTGAATATGGGCCAAACAGGATATCTGTGGTAAGCAGTTCCTGCCC
CGGCTCAGGGCCAAGAACAGATGGTCCCCAGATGCGGTCCAGCCCTCAGCAGTTTCTAGAGAACCATCAGATGTTTC
CAGGGTGCCCCAAGGACCTGAAATGACCCTGTGCCTTATTTGAACTAACCAATCAGTTCGCTTCTCGCTTCTGTTCG
CGCGCTTCTGCTCCCCGAGCTCAATAAAAGAGCCCACAACCCCTCACTCGGGGCGCCAGTCCTCCGATTGACTGAGT
CGCCCGGGTACCCGTGTATCCAATAAACCCTCTTGCAGTTGCATCCGACTTGTGGTCTCGCTGTTCCTTGGGAGGGT
CTCCTCTGAGTGATTGACTACCCGTCAGCGGGGGTCTTTCACACATGCAGCATGTATCAAAATTAATTTGGTTTTTT
TTCTTAAGTATTTACATTAAATGGCCATAGTACTTAAAGTTACATTGGCTTCCTTGAAATAAACATGGAGTATTCAG
AATGTGTCATAAATATTTCTAATTTTAAGATAGTATCTCCATTGGCTTTCTACTTTTTCTTTTATTTTTTTTTGTCC
TCTGTCTTCCATTTGTTGTTGTTGTTGTTTGTTTGTTTGTTTGTTGGTTGGTTGGTTAATTTTTTTTTAAAGATCCT
ACACTATAGTTCAAGCTAGACTATTAGCTACTCTGTAACCCAGGGTGACCTTGAAGTCATGGGTAGCCTGCTGTTTT
AGCCTTCCCACATCTAAGATTACAGGTATGAGCTATCATTTTTGGTATATTGATTGATTGATTGATTGATGTGTGTG
TGTGTGATTGTGTTTGTGTGTGTGACTGTGAAAATGTGTGTATGGGTGTGTGTGAATGTGTGTATGTATGTGTGTGT
GTGAGTGTGTGTGTGTGTGTGTGCATGTGTGTGTGTGTGACTGTGTCTATGTGTATGACTGTGTGTGTGTGTGTGTG
TGTGTGTGTGTGTGTGTGTGTGTGTGTGTTGTGAAAAAATATTCTATGGTAGTGAGAGCCAACGCTCCGGCTCAGGT
GTCAGGTTGGTTTTTGAGACAGAGTCTTTCACTTAGCTTGGAATTCACTGGCCGTCGTTTTACAACGTCGTGACTGG
GAAAACCCTGGCGTTACCCAACTTAATCGCCTTGCAGCACATCCCCCTTTCGCCAGCTGGCGTAATAGCGAAGAGGC
CCGCACCGATCGCCCTTCCCAACAGTTGCGCAGCCTGAATGGCGAATGGCGCCTGATGCGGTATTTTCTCCTTACGC
ATCTGTGCGGTATTTCACACCGCATATGGTGCACTCTCAGTACAATCTGCTCTGATGCCGCATAGTTAAGCCAGCCC
CGACACCCGCCAACACCCGCTGACGCGCCCTGACGGGCTTGTCTGCTCCCGGCATCCGCTTACAGACAAGCTGTGAC
CGTCTCCGGGAGCTGCATGTGTCAGAGGTTTTCACCGTCATCACCGAAACGCGCGAGACGAAAGGGCCTCGTGATAC
GCCTATTTTTATAGGTTAATGTCATGATAATAATGGTTTCTTAGACGTCAGGTGGCACTTTTCGGGGAAATGTGCGC
GGAACCCCTATTTGTTTATTTTTCTAAATACATTCAAATATGTATCCGCTCATGAGACAATAACCCTGATAAATGCT
TCAATAATATTGAAAAAGGAAGAGTATGAGTATTCAACATTTCCGTGTCGCCCTTATTCCCTTTTTTGCGGCATTTT
GCCTTCCTGTTTTTGCTCACCCAGAAACGCTGGTGAAAGTAAAAGATGCTGAAGATCAGTTGGGTGCACGAGTGGGT
TACATCGAACTGGATCTCAACAGCGGTAAGATCCTTGAGAGTTTTCGCCCCGAAGAACGTTTTCCAATGATGAGCAC
TTTTAAAGTTCTGCTATGTGGCGCGGTATTATCCCGTATTGACGCCGGGCAAGAGCAACTCGGTCGCCGCATACACT
ATTCTCAGAATGACTTGGTTGAGTACTCACCAGTCACAGAAAAGCATCTTACGGATGGCATGACAGTAAGAGAATTA
TGCAGTGCTGCCATAACCATGAGTGATAACACTGCGGCCAACTTACTTCTGACAACGATCGGAGGACCGAAGGAGCT
AACCGCTTTTTTGCACAACATGGGGGATCATGTAACTCGCCTTGATCGTTGGGAACCGGAGCTGAATGAAGCCATAC
CAAACGACGAGCGTGACACCACGATGCCTGTAGCAATGGCAACAACGTTGCGCAAACTATTAACTGGCGAACTACTT
ACTCTAGCTTCCCGGCAACAATTAATAGACTGGATGGAGGCGGATAAAGTTGCAGGACCACTTCTGCGCTCGGCCCT
TCCGGCTGGCTGGTTTATTGCTGATAAATCTGGAGCCGGTGAGCGTGGGTCTCGCGGTATCATTGCAGCACTGGGGC
CAGATGGTAAGCCCTCCCGTATCGTAGTTATCTACACGACGGGGAGTCAGGCAACTATGGATGAACGAAATAGACAG
ATCGCTGAGATAGGTGCCTCACTGATTAAGCATTGGTAACTGTCAGACCAAGTTTACTCATATATACTTTAGATTGA
TTTAAAACTTCATTTTTAATTTAAAAGGATCTAGGTGAAGATCCTTTTTGATAATCTCATGACCAAAATCCCTTAAC
GTGAGTTTTCGTTCCACTGAGCGTCAGACCCCGTAGAAAAGATCAAAGGATCTTCTTGAGATCCTTTTTTTCTGCGC
GTAATCTGCTGCTTGCAAACAAAAAAACCACCGCTACCAGCGGTGGTTTGTTTGCCGGATCAAGAGCTACCAACTCT
TTTTCCGAAGGTAACTGGCTTCAGCAGAGCGCAGATACCAAATACTGTTCTTCTAGTGTAGCCGTAGTTAGGCCACC
ACTTCAAGAACTCTGTAGCACCGCCTACATACCTCGCTCTGCTAATCCTGTTACCAGTGGCTGCTGCCAGTGGCGAT
AAGTCGTGTCTTACCGGGTTGGACTCAAGACGATAGTTACCGGATAAGGCGCAGCGGTCGGGCTGAACGGGGGGTTC
GTGCACACAGCCCAGCTTGGAGCGAACGACCTACACCGAACTGAGATACCTACAGCGTGAGCTATGAGAAAGCGCCA
CGCTTCCCGAAGGGAGAAAGGCGGACAGGTATCCGGTAAGCGGCAGGGTCGGAACAGGAGAGCGCACGAGGGAGCTT
CCAGGGGGAAACGCCTGGTATCTTTATAGTCCTGTCGGGTTTCGCCACCTCTGACTTGAGCGTCGATTTTTGTGATG
CTCGTCAGGGGGGCGGAGCCTATGGAAAAACGCCAGCAACGCGGCCTTTTTACGGTTCCTGGCCTTTTGCTGGCCTT
TTGCTCACATGTTCTTTCCTGCGTTATCCCCTGATTCTGTGGATAACCGTATTACCGCCTTTGAGTGAGCTGATACC
GCTCGCCGCAGCCGAACGACCGAGCGCAGCGAGTCAGTGAGCGAGGAAGCGGAAGAGCGCCCAATACGCAAACCGCC
TCTCCCCGCGCGTTGGCCGATTCATTAATGCAGCTGGCACGACAGGTTTCCCGACTGGAAAGCGGGCAGTGAGCGCA
ACGCAATTAATGTGAGTTAGCTCACTCATTAGGCACCCCAGGCTTTACACTTTATGCTTCCGGCTCGTATGTTGTGT
GGAATTGTGAGCGGATAACAATTTCACACAGGAAACAGCTATGACCATGATTACGCCAAGCTTTGCTCTTAGGAGTT
TCCTAATACATCCCAAACTCAAATATATAAAGCATTTGACTTGTTCTATGCCCTAGGGGGCGGGGGGAAGCTAAGCC
AGCTTTTTTTAACATTTAAAATGTTAATTCCATTTTAAATGCACAGATGTTTTTATTTCATAAGGGTTTCAATGTGC
ATGAATGCTGCAATATTCCTGTTACCAAAGCTAGTATAAATAAAAATAGATAAACGTGGAAATTACTTAGAGTTTCT
GTCATTAACGTTTCCTTCCTCAGTTGACAACATAAATGCGCTGCTGAGCAAGCCAGTTTGCATCTGTCAGGATCAAT
TTCCCATTATGCCAGTCATATTAATTACTAGTCAATTAGTTGATTTTTATTTTTGACATATACATGTG

One example of amino acid sequence for a vector that encodes a TNF-alpha mutant del Val1 del Pro12 and that separately encodes a CD19-specific CAR with an IgG1 hinge, CD28, and CD3zeta and that separately encodes IL15 is as follows:

(SEQ ID NO: 36)
MSTESMIRDVELAEEALPKKTGGPQGSRRCLFLSLFSFLIVAGATTLF
CLLHFGVIGPQREEFPRDLSLISPLAQARSSSRTPSDKVAHVVANPQA
EGQLQWLNRRANALLANGVELRDNQLVVPSEGLYLIYSQVLFKGQGCP
STHVLLTHTISRIAVSYQTKVNLLSAIKSPCQRETPEGAEAKPWYEPI
YLGGVFQLEKGDRLSAEINRPDYLDFAESGQVYFGIIALSRAEGRGSL
LTCGDVEENPGPMEFGLSWLFLVAILKGVQCSRDIQMTQTTSSLSASL
GDRVTISCRASQDISKYLNWYQQKPDGTVKLLIYHTSRLHSGVPSRFS
GSGSGTDYSLTISNLEQEDIATYFCQQGNTLPYTFGGGTKLELKRGGG
GSGGGGSGGGGSGGGGSEVQLQQSGPGLVAPSQSLSVTCTVSGVSLPD
YGVSWIRQPPRKGLEWLGVIWGSETTYYNSALKSRLTIIKDNSKSQVF
LKMNSLQTDDTAIYYCAKHYYYGGSYAMDYWGQGTTVTVSSYVTVSSQ
DPAEPKSPDKTHTCPPCPAPELLGGPSVFLFPPKPKDTLMISRTPEVT
CVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSF
FLYSKLTVDKSRWQQGNVFSCSVMHEALHNHYTQKSLSLSPGKKDPKF
WVLVVVGGVLACYSLLVTVAFIIFWVRSKRSRLLHSDYMNMTPRRPGP
TRKHYQPYAPPRDFAAYRSRVKFSRSADAPAYQQGQNQLYNELNLGRR
EEYDVLDKRRGRDPEMGGKPRRKNPQEGLYNELQKDKMAEAYSEIGMK
GERRRGKGHDGLYQGLSTATKDTYDALHMQALPPRGPQCTNYALLKLA
GDVESNPGPMRISKPHLRSISIQCYLCLLLNSHFLTEAGIHVFILGCF
SAGLPKTEANWVNVISDLKKIEDLIQSMHIDATLYTESDVHPSCKVTA
MKCFLLELQVISLESGDASIHDTVENLIILANNSLSSNGNVTESGCKE
CEELEEKNIKEFLQSFVHIVQMFINTS

One example of nucleic acid sequence for a vector that encodes a TNF-alpha mutant del Val1 del Pro12 and that separately encodes a CD19-specific CAR with an IgG1 hinge, DAP12, and CD3zeta and that separately encodes IL15 is as follows:

(SEQ ID NO: 16)
AATGAAAGACCCCACCTGTAGGTTTGGCAAGCTAGCTTAAGTAACGCCATTTTGCAAGGCATGGAAAAATA
CATAACTGAGAATAGAAAAGTTCAGATCAAGGTCAGGAACAGATGGAACAGCTGAATATGGGCCAAACAGGATATCT
GTGGTAAGCAGTTCCTGCCCCGGCTCAGGGCCAAGAACAGATGGAACAGCTGAATATGGGCCAAACAGGATATCTGT
GGTAAGCAGTTCCTGCCCCGGCTCAGGGCCAAGAACAGATGGTCCCCAGATGCGGTCCAGCCCTCAGCAGTTTCTAG
AGAACCATCAGATGTTTCCAGGGTGCCCCAAGGACCTGAAATGACCCTGTGCCTTATTTGAACTAACCAATCAGTTC
GCTTCTCGCTTCTGTTCGCGCGCTTATGCTCCCCGAGCTCAATAAAAGAGCCCACAACCCCTCACTCGGGGCGCCAG
TCCTCCGATTGACTGAGTCGCCCGGGTACCCGTGTATCCAATAAACCCTCTTGCAGTTGCATCCGACTTGTGGTCTC
GCTGTTCCTTGGGAGGGTCTCCTCTGAGTGATTGACTACCCGTCAGCGGGGGTCTTTCATTTGGGGGCTCGTCCGGG
ATCGGGAGACCCCTGCCCAGGGACCACCGACCCACCACCGGGAGGTAAGCTGGCCAGCAACTTATCTGTGTCTGTCC
GATTGTCTAGTGTCTATGACTGATTTTATGCGCCTGCGTCGGTACTAGTTAGCTAACTAGCTCTGTATCTGGCGGAC
CCGTGGTGGAACTGACGAGTTCGGAACACCCGGCCGCAACCCTGGGAGACGTCCCAGGGACTTCGGGGGCCGTTTTT
GTGGCCCGACCTGAGTCCTAAAATCCCGATCGTTTAGGACTCTTTGGTGCACCCCCCTTAGAGGAGGGATATGTGGT
TCTGGTAGGAGACGAGAACCTAAAACAGTTCCCGCCTCCGTCTGAATTTTTGCTTTCGGTTTGGGACCGAAGCCGCG
CCGCGCGTCTTGTCTGCTGCAGCATCGTTCTGTGTTGTCTCTGTCTGACTGTGTTTCTGTATTTGTCTGAAAATATG
GGCCCGGGCTAGCCTGTTACCACTCCCTTAAGTTTGACCTTAGGTCACTGGAAAGATGTCGAGCGGATCGCTCACAA
CCAGTCGGTAGATGTCAAGAAGAGACGTTGGGTTACCTTCTGCTCTGCAGAATGGCCAACCTTTAACGTCGGATGGC
CGCGAGACGGCACCTTTAACCGAGACCTCATCACCCAGGTTAAGATCAAGGTCTTTTCACCTGGCCCGCATGGACAC
CCAGACCAGGTGGGGTACATCGTGACCTGGGAAGCCTTGGCTTTTGACCCCCCTCCCTGGGTCAAGCCCTTTGTACA
CCCTAAGCCTCCGCCTCCTCTTCCTCCATCCGCCCCGTCTCTCCCCCTTGAACCTCCTCGTTCGACCCCGCCTCGAT
CCTCCCTTTATCCAGCCCTCACTCCTTCTCTAGGCGCCCCCATATGGCCATATGAGATCTTATATGGGGCACCCCCG
CCCCTTGTAAACTTCCCTGACCCTGACATGACAAGAGTTACTAACAGCCCCTCTCTCCAAGCTCACTTACAGGCTCT
CTACTTAGTCCAGCACGAAGTCTGGAGACCTCTGGCGGCAGCCTACCAAGAACAACTGGACCGACCGGTGGTACCTC
ACCCTTACCGAGTCGGCGACACAGTGTGGGTCCGCCGACACCAGACTAAGAACCTAGAACCTCGCTGGAAAGGACCT
TACACAGTCCTGCTGACCACCCCCACCGCCCTCAAAGTAGACGGCATCGCAGCTTGGATACACGCCGCCCACGTGAA
GGCTGCCGACCCCGGGGGTGGACCATCCTCTAGACTGCCATGCTCGAGATGAGCACTGAAAGCATGATCCGGGACGT
GGAGCTGGCCGAGGAGGCGCTCCCCAAGAAGACAGGGGGGCCCCAGGGCTCCAGGCGGTGCTTGTTCCTCAGCCTCT
TCTCCTTCCTGATCGTGGCAGGCGCCACCACGCTCTTCTGCCTGCTGCACTTTGGAGTGATCGGCCCCCAGAGGGAA
GAGTTCCCCAGGGACCTCTCTCTAATCAGCCCTCTGGCCCAGGCAAGATCATCTTCTCGAACCCCGAGTGACAAGGT
AGCCCATGTTGTAGCAAACCCTCAAGCTGAGGGGCAGCTCCAGTGGCTGAACCGCCGGGCCAATGCCCTCCTGGCCA
ATGGCGTGGAGCTGAGAGATAACCAGCTGGTGGTGCCATCAGAGGGCCTGTACCTCATCTACTCCCAGGTCCTCTTC
AAGGGCCAAGGCTGCCCCTCCACCCATGTGCTCCTCACCCACACCATCAGCCGCATCGCCGTCTCCTACCAGACCAA
GGTCAACCTCCTCTCTGCCATCAAGAGCCCCTGCCAGAGGGAGACCCCAGAGGGGGCTGAGGCCAAGCCCTGGTATG
AGCCCATCTATCTGGGAGGGGTCTTCCAGCTGGAGAAGGGTGACCGACTCAGCGCTGAGATCAATCGGCCCGACTAT
CTCGACTTTGCCGAGTCTGGGCAGGTCTACTTTGGGATCATTGCCCTGTCGCGAGCCGAGGGCAGGGGAAGTCTTCT
AACATGCGGGGACGTGGAGGAAAATCCCGGGCCCATGGAGTTTGGGCTGAGCTGGCTTTTTCTTGTGGCTATTTTAA
AAGGTGTCCAGTGCTCTAGAGACATCCAGATGACACAGACTACATCCTCCCTGTCTGCCTCTCTGGGAGACAGAGTC
ACCATCAGTTGCAGGGCAAGTCAGGACATTAGTAAATATTTAAATTGGTATCAGCAGAAACCAGATGGAACTGTTAA
ACTCCTGATCTACCATACATCAAGATTACACTCAGGAGTCCCATCAAGGTTCAGTGGCAGTGGGTCTGGAACAGATT
ATTCTCTCACCATTAGCAACCTGGAGCAAGAAGATATTGCCACTTACTTTTGCCAACAGGGTAATACGCTTCCGTAC
ACGTTCGGAGGGGGGACCAAGCTGGAGCTGAAACGTGGTGGTGGTGGTTCTGGTGGTGGTGGTTCTGGCGGCGGCGG
CTCCGGTGGTGGTGGATCCGAGGTGCAGCTGCAGCAGTCTGGACCTGGCCTGGTGGCGCCCTCACAGAGCCTGTCCG
TCACATGCACTGTCTCAGGGGTCTCATTACCCGACTATGGTGTAAGCTGGATTCGCCAGCCTCCACGAAAGGGTCTG
GAGTGGCTGGGAGTAATATGGGGTAGTGAAACCACATACTATAATTCAGCTCTCAAATCCAGACTGACCATCATCAA
GGACAACTCCAAGAGCCAAGTTTTCTTAAAAATGAACAGTCTGCAAACTGATGACACAGCCATTTACTACTGTGCCA
AACATTATTACTACGGTGGTAGCTATGCTATGGACTACTGGGGCCAAGGGACCACGGTCACTGTCTCCTCGTACGTC
ACTGTCTCTTCACAGGATCCCGCCGAGCCCAAATCTCCTGACAAAACTCACACATGCCCACCGTGCCCAGCACCTGA
ACTCCTGGGGGGACCGTCAGTCTTCCTCTTCCCCCCAAAACCCAAGGACACCCTCATGATCTCCCGGACCCCTGAGG
TCACATGCGTGGTGGTGGACGTGAGCCACGAAGACCCTGAGGTCAAGTTCAACTGGTACGTGGACGGCGTGGAGGTG
CATAATGCCAAGACAAAGCCGCGGGAGGAGCAGTACAACAGCACGTACCGTGTGGTCAGCGTCCTCACCGTCCTGCA
CCAGGACTGGCTGAATGGCAAGGAGTACAAGTGCAAGGTCTCCAACAAAGCCCTCCCAGCCCCCATCGAGAAAACCA
TCTCCAAAGCCAAAGGGCAGCCCCGAGAACCACAGGTGTACACCCTGCCCCCATCCCGGGATGAGCTGACCAAGAAC
CAGGTCAGCCTGACCTGCCTGGTCAAAGGCTTCTATCCCAGCGACATCGCCGTGGAGTGGGAGAGCAATGGGCAACC
GGAGAACAACTACAAGACCACGCCTCCCGTGCTGGACTCCGACGGCTCCTTCTTCCTCTACAGCAAGCTCACCGTGG
ACAAGAGCAGGTGGCAGCAGGGGAACGTCTTCTCATGCTCCGTGATGCATGAGGCTCTGCACAACCACTACACGCAG
AAGAGCCTCTCCCTGTCTCCGGGTAAAAAAGATCCCAAATTTTGGGGCGTGCTGGCCGGAATCGTGATGGGCGACCT
GGTGCTGACAGTGCTGATCGCCCTGGCTGTGTACTTCCTGGGCAGACTGGTGCCCAGAGGAAGAGGCGCTGCCGAAG
CCGCCACCCGGAAGCAGAGAATCACCGAGACAGAGAGCCCCTATCAGGAACTGCAGGGCCAGCGGAGCGACGTGTAC
AGCGACCTGAATACCCAGCGGCCCTACTACAAACGCGGACCGCAGTGTACTAATTATGCTCTCTTGAAATTGGCTGG
AGATGTTGAGAGCAATCCCGGGCCCATGCGCATTAGCAAGCCCCACCTGCGGAGCATCAGCATCCAGTGCTACCTGT
GCCTGCTGCTGAACAGCCACTTCCTGACCGAGGCCGGCATCCACGTGTTCATCCTGGGCTGCTTCAGCGCCGGACTG
CCCAAGACCGAGGCCAACTGGGTGAACGTGATCAGCGACCTGAAGAAGATCGAGGACCTGATCCAGAGCATGCACAT
CGACGCCACCCTGTACACCGAGAGCGACGTGCACCCCAGCTGCAAGGTGACCGCCATGAAGTGCTTTCTGCTGGAAC
TGCAGGTGATCAGCCTGGAAAGCGGCGACGCCAGCATCCACGACACCGTGGAGAACCTGATCATCCTGGCCAACAAC
AGCCTGAGCAGCAACGGCAACGTGACCGAGAGCGGCTGCAAAGAGTGCGAGGAACTGGAAGAGAAGAACATCAAAGA
GTTTCTGCAGAGCTTCGTGCACATCGTGCAGATGTTCATCAACACCAGCTGACAATTGCGCGTCATCATCGATCCGG
ATTAGTCCAATTTGTTAAAGACAGGATATCAGTGGTCCAGGCTCTAGTTTTGACTCAACAATATCACCAGCTGAAGC
CTATAGAGTACGAGCCATAGATAAAATAAAAGATTTTATTTAGTCTCCAGAAAAAGGGGGGAATGAAAGACCCCACC
TGTAGGTTTGGCAAGCTAGCTTAAGTAACGCCATTTTGCAAGGCATGGAAAAATACATAACTGAGAATAGAGAAGTT
CAGATCAAGGTCAGGAACAGATGGAACAGCTGAATATGGGCCAAACAGGATATCTGTGGTAAGCAGTTCCTGCCCCG
GCTCAGGGCCAAGAACAGATGGAACAGCTGAATATGGGCCAAACAGGATATCTGTGGTAAGCAGTTCCTGCCCCGGC
TCAGGGCCAAGAACAGATGGTCCCCAGATGCGGTCCAGCCCTCAGCAGTTTCTAGAGAACCATCAGATGTTTCCAGG
GTGCCCCAAGGACCTGAAATGACCCTGTGCCTTATTTGAACTAACCAATCAGTTCGCTTCTCGCTTCTGTTCGCGCG
CTTCTGCTCCCCGAGCTCAATAAAAGAGCCCACAACCCCTCACTCGGGGCGCCAGTCCTCCGATTGACTGAGTCGCC
CGGGTACCCGTGTATCCAATAAACCCTCTTGCAGTTGCATCCGACTTGTGGTCTCGCTGTTCCTTGGGAGGGTCTCC
TCTGAGTGATTGACTACCCGTCAGCGGGGGTCTTTCACACATGCAGCATGTATCAAAATTAATTTGGTTTTTTTTCT
TAAGTATTTACATTAAATGGCCATAGTACTTAAAGTTACATTGGCTTCCTTGAAATAAACATGGAGTATTCAGAATG
TGTCATAAATATTTCTAATTTTAAGATAGTATCTCCATTGGCTTTCTACTTTTTCTTTTATTTTTTTTTGTCCTCTG
TCTTCCATTTGTTGTTGTTGTTGTTTGTTTGTTTGTTTGTTGGTTGGTTGGTTAATTTTTTTTTAAAGATCCTACAC
TATAGTTCAAGCTAGACTATTAGCTACTCTGTAACCCAGGGTGACCTTGAAGTCATGGGTAGCCTGCTGTTTTAGCC
TTCCCACATCTAAGATTACAGGTATGAGCTATCATTTTTGGTATATTGATTGATTGATTGATTGATGTGTGTGTGTG
TGATTGTGTTTGTGTGTGTGACTGTGAAAATGTGTGTATGGGTGTGTGTGAATGTGTGTATGTATGTGTGTGTGTGA
GTGTGTGTGTGTGTGTGTGCATGTGTGTGTGTGTGACTGTGTCTATGTGTATGACTGTGTGTGTGTGTGTGTGTGTG
TGTGTGTGTGTGTGTGTGTGTGTGTTGTGAAAAAATATTCTATGGTAGTGAGAGCCAACGCTCCGGCTCAGGTGTCA
GGTTGGTTTTTGAGACAGAGTCTTTCACTTAGCTTGGAATTCACTGGCCGTCGTTTTACAACGTCGTGACTGGGAAA
ACCCTGGCGTTACCCAACTTAATCGCCTTGCAGCACATCCCCCTTTCGCCAGCTGGCGTAATAGCGAAGAGGCCCGC
ACCGATCGCCCTTCCCAACAGTTGCGCAGCCTGAATGGCGAATGGCGCCTGATGCGGTATTTTCTCCTTACGCATCT
GTGCGGTATTTCACACCGCATATGGTGCACTCTCAGTACAATCTGCTCTGATGCCGCATAGTTAAGCCAGCCCCGAC
ACCCGCCAACACCCGCTGACGCGCCCTGACGGGCTTGTCTGCTCCCGGCATCCGCTTACAGACAAGCTGTGACCGTC
TCCGGGAGCTGCATGTGTCAGAGGTTTTCACCGTCATCACCGAAACGCGCGAGACGAAAGGGCCTCGTGATACGCCT
ATTTTTATAGGTTAATGTCATGATAATAATGGTTTCTTAGACGTCAGGTGGCACTTTTCGGGGAAATGTGCGCGGAA
CCCCTATTTGTTTATTTTTCTAAATACATTCAAATATGTATCCGCTCATGAGACAATAACCCTGATAAATGCTTCAA
TAATATTGAAAAAGGAAGAGTATGAGTATTCAACATTTCCGTGTCGCCCTTATTCCCTTTTTTGCGGCATTTTGCCT
TCCTGTTTTTGCTCACCCAGAAACGCTGGTGAAAGTAAAAGATGCTGAAGATCAGTTGGGTGCACGAGTGGGTTACA
TCGAACTGGATCTCAACAGCGGTAAGATCCTTGAGAGTTTTCGCCCCGAAGAACGTTTTCCAATGATGAGCACTTTT
AAAGTTCTGCTATGTGGCGCGGTATTATCCCGTATTGACGCCGGGCAAGAGCAACTCGGTCGCCGCATACACTATTC
TCAGAATGACTTGGTTGAGTACTCACCAGTCACAGAAAAGCATCTTACGGATGGCATGACAGTAAGAGAATTATGCA
GTGCTGCCATAACCATGAGTGATAACACTGCGGCCAACTTACTTCTGACAACGATCGGAGGACCGAAGGAGCTAACC
GCTTTTTTGCACAACATGGGGGATCATGTAACTCGCCTTGATCGTTGGGAACCGGAGCTGAATGAAGCCATACCAAA
CGACGAGCGTGACACCACGATGCCTGTAGCAATGGCAACAACGTTGCGCAAACTATTAACTGGCGAACTACTTACTC
TAGCTTCCCGGCAACAATTAATAGACTGGATGGAGGCGGATAAAGTTGCAGGACCACTTCTGCGCTCGGCCCTTCCG
GCTGGCTGGTTTATTGCTGATAAATCTGGAGCCGGTGAGCGTGGGTCTCGCGGTATCATTGCAGCACTGGGGCCAGA
TGGTAAGCCCTCCCGTATCGTAGTTATCTACACGACGGGGAGTCAGGCAACTATGGATGAACGAAATAGACAGATCG
CTGAGATAGGTGCCTCACTGATTAAGCATTGGTAACTGTCAGACCAAGTTTACTCATATATACTTTAGATTGATTTA
AAACTTCATTTTTAATTTAAAAGGATCTAGGTGAAGATCCTTTTTGATAATCTCATGACCAAAATCCCTTAACGTGA
GTTTTCGTTCCACTGAGCGTCAGACCCCGTAGAAAAGATCAAAGGATCTTCTTGAGATCCTTTTTTTCTGCGCGTAA
TCTGCTGCTTGCAAACAAAAAAACCACCGCTACCAGCGGTGGTTTGTTTGCCGGATCAAGAGCTACCAACTCTTTTT
CCGAAGGTAACTGGCTTCAGCAGAGCGCAGATACCAAATACTGTTCTTCTAGTGTAGCCGTAGTTAGGCCACCACTT
CAAGAACTCTGTAGCACCGCCTACATACCTCGCTCTGCTAATCCTGTTACCAGTGGCTGCTGCCAGTGGCGATAAGT
CGTGTCTTACCGGGTTGGACTCAAGACGATAGTTACCGGATAAGGCGCAGCGGTCGGGCTGAACGGGGGGTTCGTGC
ACACAGCCCAGCTTGGAGCGAACGACCTACACCGAACTGAGATACCTACAGCGTGAGCTATGAGAAAGCGCCACGCT
TCCCGAAGGGAGAAAGGCGGACAGGTATCCGGTAAGCGGCAGGGTCGGAACAGGAGAGCGCACGAGGGAGCTTCCAG
GGGGAAACGCCTGGTATCTTTATAGTCCTGTCGGGTTTCGCCACCTCTGACTTGAGCGTCGATTTTTGTGATGCTCG
TCAGGGGGGCGGAGCCTATGGAAAAACGCCAGCAACGCGGCCTTTTTACGGTTCCTGGCCTTTTGCTGGCCTTTTGC
TCACATGTTCTTTCCTGCGTTATCCCCTGATTCTGTGGATAACCGTATTACCGCCTTTGAGTGAGCTGATACCGCTC
GCCGCAGCCGAACGACCGAGCGCAGCGAGTCAGTGAGCGAGGAAGCGGAAGAGCGCCCAATACGCAAACCGCCTCTC
CCCGCGCGTTGGCCGATTCATTAATGCAGCTGGCACGACAGGTTTCCCGACTGGAAAGCGGGCAGTGAGCGCAACGC
AATTAATGTGAGTTAGCTCACTCATTAGGCACCCCAGGCTTTACACTTTATGCTTCCGGCTCGTATGTTGTGTGGAA
TTGTGAGCGGATAACAATTTCACACAGGAAACAGCTATGACCATGATTACGCCAAGCTTTGCTCTTAGGAGTTTCCT
AATACATCCCAAACTCAAATATATAAAGCATTTGACTTGTTCTATGCCCTAGGGGGCGGGGGGAAGCTAAGCCAGCT
TTTTTTAACATTTAAAATGTTAATTCCATTTTAAATGCACAGATGTTTTTATTTCATAAGGGTTTCAATGTGCATGA
ATGCTGCAATATTCCTGTTACCAAAGCTAGTATAAATAAAAATAGATAAACGTGGAAATTACTTAGAGTTTCTGTCA
TTAACGTTTCCTTCCTCAGTTGACAACATAAATGCGCTGCTGAGCAAGCCAGTTTGCATCTGTCAGGATCAATTTCC
CATTATGCCAGTCATATTAATTACTAGTCAATTAGTTGATTTTTATTTTTGACATATACATGTG.

One example of amino acid sequence for a vector that encodes a TNF-alpha mutant del Val1 del Pro12 and that separately encodes a CD19-specific CAR with an IgG1 hinge, DAP12, and CD3zeta and that separately encodes IL15 is as follows:

(SEQ ID NO: 37)
MSTESMIRDVELAEEALPKKTGGPQGSRRCLFLSLFSFLIVAGATTLF
CLLHFGVIGPQREEFPRDLSLISPLAQARSSSRTPSDKVAHVVANPQA
EGQLQWLNRRANALLANGVELRDNQLVVPSEGLYLIYSQVLFKGQGCP
STHVLLTHTISRIAVSYQTKVNLLSAIKSPCQRETPEGAEAKPWYEPI
YLGGVFQLEKGDRLSAEINRPDYLDFAESGQVYFGIIALSRAEGRGSL
LTCGDVEENPGPMEFGLSWLFLVAILKGVQCSRDIQMTQTTSSLSASL
GDRVTISCRASQDISKYLNWYQQKPDGTVKLLIYHTSRLHSGVPSRFS
GSGSGTDYSLTISNLEQEDIATYFCQQGNTLPYTFGGGTKLELKRGGG
GSGGGGSGGGGSGGGGSEVQLQQSGPGLVAPSQSLSVTCTVSGVSLPD
YGVSWIRQPPRKGLEWLGVIWGSETTYYNSALKSRLTIIKDNSKSQVF
LKMNSLQTDDTAIYYCAKHYYYGGSYAMDYWGQGTTVTVSSYVTVSSQ
DPAEPKSPDKTHTCPPCPAPELLGGPSVFLFPPKPKDTLMISRTPEVT
CVVVDVSHEDPEVKFNWYVDGVEVHNAKTKPREEQYNSTYRVVSVLTV
LHQDWLNGKEYKCKVSNKALPAPIEKTISKAKGQPREPQVYTLPPSRD
ELTKNQVSLTCLVKGFYPSDIAVEWESNGQPENNYKTTPPVLDSDGSF
FLYSKLTVDKSRWQQGNVFSCSVMHEALHNHYTQKSLSLSPGKKDPKF
WGVLAGIVMGDLVLTVLIALAVYFLGRLVPRGRGAAEAATRKQRITET
ESPYQELQGQRSDVYSDLNTQRPYYKRVKFSRSADAPAYQQGQNQLYN
ELNLGRREEYDVLDKRRGRDPEMGGKPRRKNPQEGLYNELQKDKMAEA
YSEIGMKGERRRGKGHDGLYQGLSTATKDTYDALHMQALPPRGPQCTN
YALLKLAGDVESNPGPMRISKPHLRSISIQCYLCLLLNSHFLTEAGIH
VFILGCFSAGLPKTEANWVNVISDLKKIEDLIQSMHIDATLYTESDVH
PSCKVTAMKCFLLELQVISLESGDASIHDTVENLIILANNSLSSNGNV
TESGCKECEELEEKNIKEFLQSFVHIVQMFINTS

### V. Cells

Embodiments of the disclosure encompass cells that express one or more TNF-alpha mutants as encompassed herein. The cell comprises a recombinant nucleic acid that encodes one or more engineered nonsecretable, membrane bound TNF-alpha mutant polypeptides, in specific embodiments. In specific embodiments, in addition to expressing one or more TNF-alpha mutant polypeptides, the cell also comprises a nucleic acid that encodes one or more therapeutic gene products. The nucleic acids may be vectors of any kind. The nucleic acid that encodes the one or more TNF-alpha mutant polypeptides may or may not be the same nucleic acid molecule that encodes the one or more therapeutic gene products.

The cells of the disclosure may be of any kind, including at least T-cells, NK cells, NKT cells, iNKT cells, macrophages, B cells, MSCs, or stem cells of any kind, including at least hematopoietic stem cells, pluripotent embryonic stem cells or embryonic stem cells.

The cells may be obtained from an individual directly or may be obtained from a depository or other storage facility. The cells as therapy may be autologous or allogeneic with respect to the individual to which the cells are provided as therapy.

The cells may be from an individual in need of therapy for a medical condition, and following their manipulation to express the TNF-alpha mutant and therapeutic gene product (using standard techniques for transduction and expansion for adoptive cell therapy, for example), they may be provided back to the individual from which they were originally sourced. In some cases, the cells are stored for later use for the individual or another individual.

The cells that harbor the one or more engineered receptors and that may be needed to be eliminated by the resident TNF-alpha suicide gene may be of any kind. In specific embodiments the cells are immune cells or stem cells, including those that are being utilized for adoptive cell therapy, for example. The immune cells may be T-cells, NK cells, NKT cells, iNKT cells, B cells, and so forth. The cells may be comprised in a population of cells, and that population may have a majority that are transduced with one or more TNF-alpha mutant suicide genes or both of one or more engineered receptors and one or more TNF-alpha mutant suicide genes. A cell population may comprise 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100% of cells that are transduced with one or more TNF-alpha mutant suicide genes and, optionally, one or more engineered receptors. The TNF-alpha mutant(s) and the engineered receptor(s) are separate polypeptides.

The cells may be produced with the TNF-alpha mutant suicide gene for the intent of being modular with respect to a specific purpose. For example, cells may be generated, including for commercial distribution, expressing a TNF-alpha mutant (or distributed with a nucleic acid that encodes the mutant for subsequent transduction), and a user may modify them to express one or more therapeutic genes of interest dependent upon their intended purpose(s). As only one example, an individual interested in treating CD5-positive cancer may obtain or generate the TNF-alpha mutant-expressing cells and modify them to express a CAR comprising a CD5-specific scFv. Alternatively, an individual interested in treating CD5-positive cancer may obtain cells to be transduced, obtain a vector that encodes the TNF-alpha mutant, and modify the vector also to encode a CD5-specific CAR, followed by subsequent transduction of the cells. Either of those embodiments may be applied to any other cancer antigen than CD5.

In particular embodiments, the genome of the transduced cells expressing the TNF-alpha mutant may be modified. The genome may be modified in any manner, but in specific embodiments the genome is modified by CRISPR gene editing, for example. The genome of the cells may be modified to enhance effectiveness of the TNF-alpha mutant as a suicide gene, to enhance effectiveness of use of the therapeutic gene product, or for another purpose. Specific examples of genes that may be modified in the cells includes the following: knockout of ADAM13/TACE, increase resistance of TNF-alpha mutant expressing cells to the tumor microenvironment such as TGF-beta receptor 1 or 2, IDO, checkpoint molecules such as PD1, TIGIT, KLRG1, TIM3, etc.

### VI. Use of TNF-Alpha Mutants as a Suicide Gene

In particular embodiments, the cells for which the TNF-alpha mutant suicide gene are employed are cells that have the potential to be deleterious, for example for an individual exposed to the cells in vivo. The cells may be toxic to an individual upon delivery or thereafter, and therefore a need to be able to eliminate the cells may be consistently present for the cells. For instance, any type of cell therapy for use in an individual in vivo would be able to employ the disclosed TNF-alpha mutants in the cells, allowing the cell therapy to be terminated when desired. The cell therapy may be subject to utilization of the TNF-alpha mutant suicide gene when an individual receiving the cell therapy and/or having received the cell therapy shows one or more symptoms of one or more adverse events, such as cytokine release syndrome, neurotoxicity, anaphylaxis/allergy, and/or on-target/off tumor toxicities (as examples) or is considered at risk for having the one or more symptoms, including imminently. The use of the TNF-alpha mutant as a suicide gene may be part of a planned protocol for a therapy or may be used only upon a recognized need for its use. In some cases the cell therapy is terminated by use of agent(s) that targets the TNF-alpha suicide gene because the therapy is no longer required.

The cells for which the TNF-alpha suicide gene is utilized may be cells engineered for cell therapy for mammals, in particular embodiments. In such cases, the cell therapy may be of any kind and the cells may be of any kind. In specific embodiments, the cells are immune cells or stem cells that have been engineered to express one or more therapeutic gene products. In specific embodiments, the cells are cells that are transduced with one or more engineered receptors for the cells. The engineered receptors may impart a therapeutic characteristic for the cells upon targeting, such as by binding to, a ligand for the receptor. In specific embodiments, the engineered receptor is non-native and made by the hand of man. The engineered receptor may be of any kind including a T-cell receptor, a chimeric antigen receptor (CAR), chemokine receptor, cytokine receptor, homing receptor, gene-edited cells, or a combination thereof. The engineered receptors may be engineered to be able to bind, such as target, a specific antigen, including at least a tumor antigen, as an example. The engineered receptors may be bi-specific or multi-specific for more than one antigen, in some cases, allowing the transduced cells to bind through the engineered receptor to cells that express the multiple antigens.

In particular embodiments, upon delivering an effective amount of one or more agents to bind to the TNF-alpha mutant-expressing cells, the majority of TNF-alpha mutant-expressing cells are eliminated. In specific embodiments, greater than 50%, 55%, 60%, 65%, 70%, 75%, 80%, 85%, 90%, 95%, 96%, 97%, 98%, or 99% of cells expressing the TNF-alpha mutants are eliminated in an individual. Following recognition of a need to eliminate the cells, the delivery of the agent(s) to the individual may continue until one or more symptoms are no longer present or until a sufficient number of cells have been eliminated. The cell numbers in the individual may be monitored using the TNF-alpha mutants as markers.

Embodiments of methods of the disclosure may comprise a first step of providing an effective amount of cell therapy to an individual in need thereof, wherein the cells comprise one or more nonsecretable TNF-alpha mutants; and, a second step of eliminating the cells using the TNF-alpha mutant(s) as suicide genes (directly or indirectly through cell death by any mechanism). The second step may be instigated upon onset of at least one adverse event for the individual, and that adverse event may be recognized by any means, including upon routine monitoring that may or may not be continuous from the beginning of the cell therapy. The adverse event(s) may be detected upon examination and/or testing. In cases wherein the individual has cytokine release syndrome (which may also be referred to as cytokine storm), the individual may have elevated inflammatory cytokine(s) (merely as examples: interferon-gamma, granulocyte macrophage colony-stimulating factor, IL-10, IL-6 and TNF-alpha); fever; fatigue; hypotension; hypoxia, tachycardia; nausea; capillary leak; cardiac/renal/hepatic dysfunction; or a combination thereof, for example. In cases wherein the individual has neurotoxicity, the individual may have confusion, delirium, aplasia, and/or seizures. In some cases, the individual is tested for a marker associated with onset and/or severity of cytokine release syndrome, such as C-reactive protein, IL-6, TNF-alpha, and/or ferritin

In additional embodiments, administration of one or more agents that bind the nonsecretable TNF-α during cytokine release syndrome or neurotoxicity, for example, have the added benefit of neutralizing the high levels of soluble TNF-alpha that contribute to the toxicity of the therapy. Soluble TNF-alpha is released at high levels during cytokine release syndrome and is a mediator of toxicity with CAR T-cell therapies. In such cases, the administration of TNF-alpha antibodies encompassed herein have a dual beneficial effect—i.e. selective deletion of the TNF-alpha mutant-expressing cells as well as neutralizing soluble TNF-alpha causing toxicity. Thus, embodiments of the disclosure encompass methods of eliminating or reducing the severity of cytokine release syndrome in an individual receiving, or who has received, adoptive cell therapy in which the cells express a nonsecretable TNF-alpha mutant, comprising the step of providing an effective amount of an agent that binds the nonsecretable TNF-alpha mutant, said agent causing in the individual (a) elimination of at least some of the cells of the cell therapy; and (b) reduction in levels of soluble TNF-alpha.

Embodiments of the disclosure include methods of reducing the effects of cytokine release syndrome in an individual that has received or who is receiving cell therapy with cells that express a nonsecretable TNF-alpha mutant, comprising the step of providing an effective amount of one or more agents that bind the mutant to cause in the individual (a) elimination of at least some of the cells of the cell therapy; and (b) reduction in the level of soluble TNF-alpha.

When the need arises for the TNF-alpha suicide gene to be utilized, the individual is provided an effective amount of one or more inhibitors that are able to inhibit, such as by binding directly, the TNF-alpha mutant on the surface of the cells. The inhibitor(s) may be provided to the individual systemically and/or locally in some embodiments. The inhibitor may be a polypeptide (such as an antibody), a nucleic acid, a small molecule (for example, a xanthine derivative), a peptide, or a combination thereof. In specific embodiments, the antibodies are FDA-approved. When the inhibitor is an antibody, the inhibitor may be a monoclonal antibody in at least some cases. When mixtures of antibodies are employed, one or more antibodies in the mixture may be a monoclonal antibody. Examples of small molecule TNF-alpha inhibitors include small molecules such as are described in U.S. Pat. No. 5,118,500, which is incorporated by reference herein in its entirety. Examples of polypeptide TNF-alpha inhibitors include polypeptides, such as those described in U.S. Pat. No. 6,143,866, which is incorporated by reference herein in its entirety.

In particular embodiments, at least one antibody is utilized to target the TNF-alpha mutant to trigger its activity as a suicide gene. Examples of antibodies includes at least Adalimumab, Adalimumab-atto, Certolizumab pegol, Etanercept, Etanercept-szzs, Golimumab, Infliximab, Infliximab-dyyb, or a mixture thereof, for example.

Embodiments of the disclosure include methods of reducing the risk of toxicity of a cell therapy for an individual by modifying cells of a cell therapy to express a nonsecretable TNF-alpha mutant. The cell therapy is for cancer, in specific embodiments, and it may comprise an engineered receptor that targets an antigen, including a cancer antigen.

In particular embodiments, in addition to the inventive cell therapy of the disclosure, the individual may have been provided, may be provided, and/or may will be provided an additional therapy for the medical condition. In cases wherein the medical condition is cancer, the individual may be provided one or more of surgery, radiation, immunotherapy (other than the cell therapy of the present disclosure), hormone therapy, gene therapy, chemotherapy, and so forth.

In cases wherein the individual being treated with the cell therapy of the disclosure has cancer, the individual may have any type of cancer. The individual may have leukemia, lymphoma, myeloma, brain cancer, lung cancer, breast cancer, colon cancer, endometrium cancer, cervical cancer, ovarian cancer, testicular cancer, bone cancer, skin cancer, kidney cancer, liver cancer, stomach cancer, spleen cancer, thyroid cancer, head and neck cancer, gall bladder cancer, and so forth.

### VII. Kits of the Disclosure

Any of the compositions described herein may be comprised in a kit. In a non-limiting example, cells, reagents to produce cells, vectors, and reagents to produce vectors and components thereof may be comprised in a kit. In certain embodiments, alpha-beta T-cells, gamma-delta T cells, NK cells, NKT cells, iNKT cells, B cells, or stem cells may be comprised in a kit. Such a kit may or may not have one or more reagents for manipulation of cells. Such reagents include small molecules, proteins, nucleic acids, antibodies, buffers, primers, nucleotides, salts, and/or a combination thereof, for example. Nucleotides that encode one or more TNF-alpha mutants, engineered receptors, or cytokines may be included in the kit. Proteins, such as cytokines or antibodies, including monoclonal antibodies, may be included in the kit. Nucleotides that encode components of engineered receptors, such as chimeric antigen receptors or T-cell receptors may be included in the kit, including reagents to generate same.

In particular aspects, the kit comprises the cell therapy of the disclosure and also another cancer therapy. In some cases, the kit, in addition to the cell therapy embodiments, also includes a second cancer therapy, such as chemotherapy, hormone therapy, and/or immunotherapy, for example. The kit(s) may be tailored to a particular cancer for an individual and comprise respective second cancer therapies for the individual.

The kits may comprise suitably aliquoted compositions of the present disclosure. The components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also may generally contain a second, third or other additional container into which the additional components may be separately placed. However, various combinations of components may be comprised in a vial. The kits of the present invention also will typically include a means for containing the composition and any other reagent containers in close confinement for commercial sale. Such containers may include injection or blow-molded plastic containers into which the desired vials are retained.

### EXAMPLES

The following examples are included to demonstrate preferred embodiments of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

### TNF-Alpha Suicide Gene

The present disclosure provides a marker moiety and a suicide moiety for cell therapy, based on uncleavable mutants of the 26 kd tumor necrosis factor alpha (TNF-α) that is normally processed to a 17 kD component. There are a number of advantages to using this approach. FIG. 1 shows an example of an experimental plan to mutagenize TNF-alpha to ablate membrane cutting sites. As described by Perez et al. (1990), the right panel of FIG. 1 illustrates three exemplary TNF-alpha mutants that render the TNF-alpha mutant to be uncleavable: (1) deletion of amino acid residues 1-12 of the 17 kD TNF; (2) deletion of amino acid residues 1 and 12 of the 17 kD TNF; and (3) deletion of amino acid residues 1 and 13 of the 17 kD TNF. The left panel of FIG. 1 provides examples of primers for site-directed mutagenesis as an example to generate the mutants.

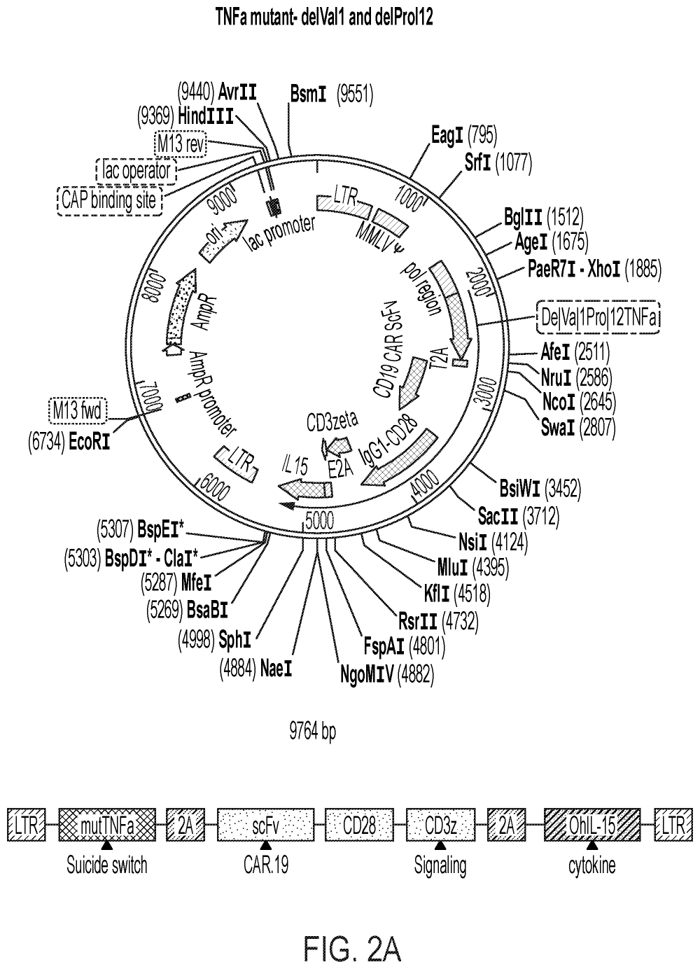

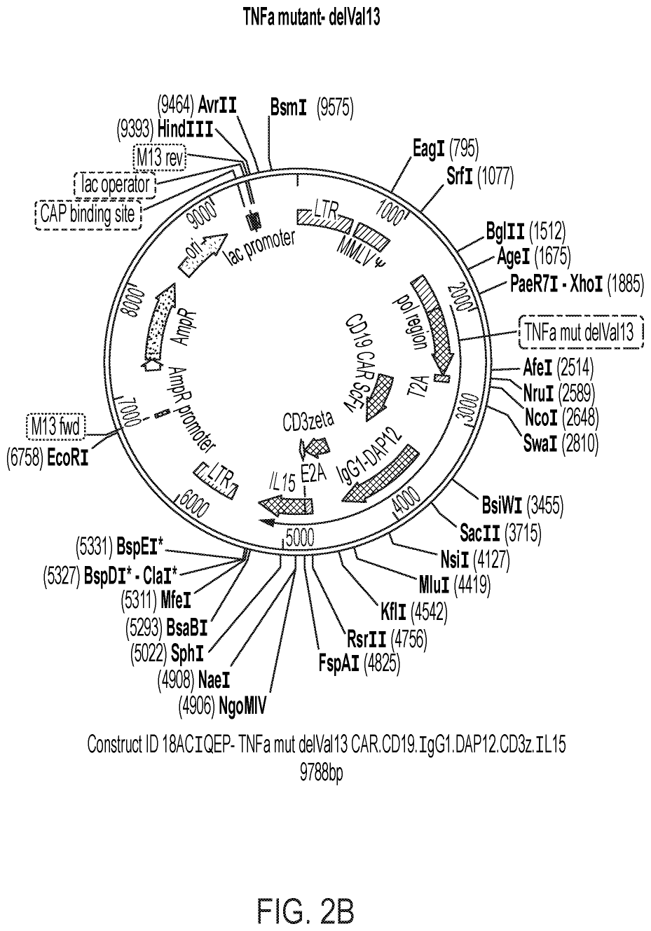

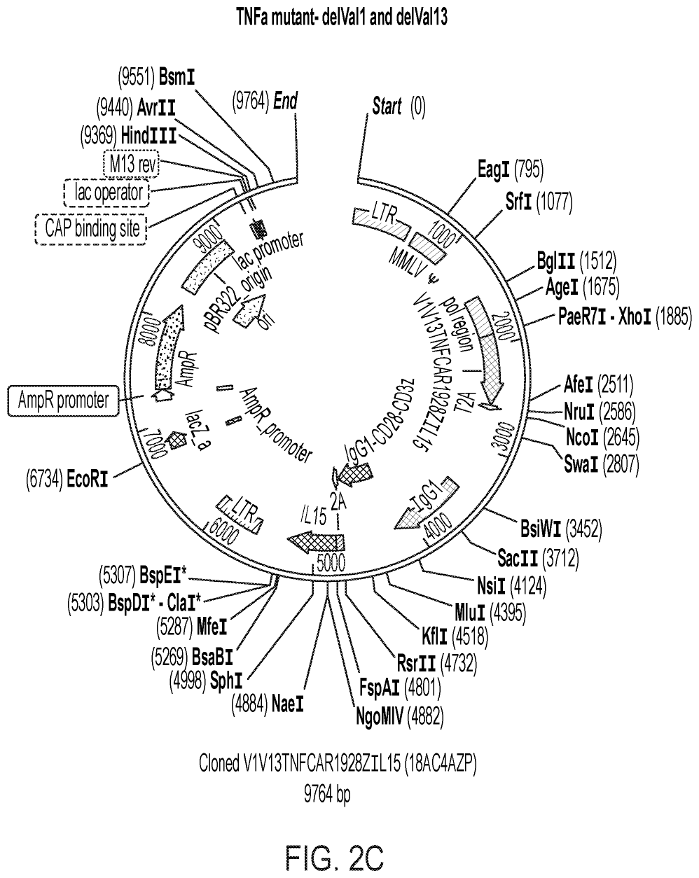

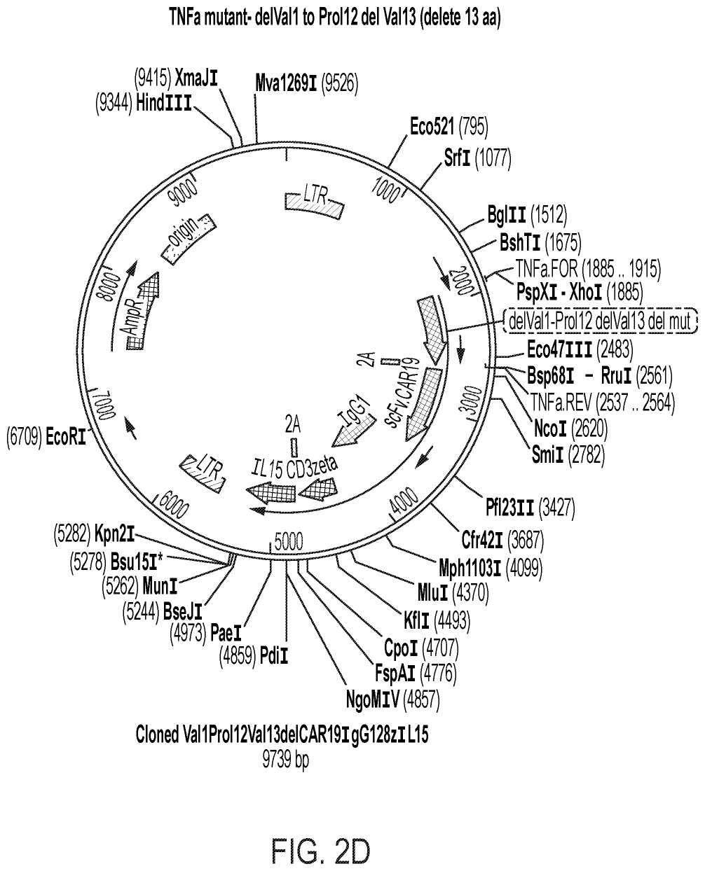

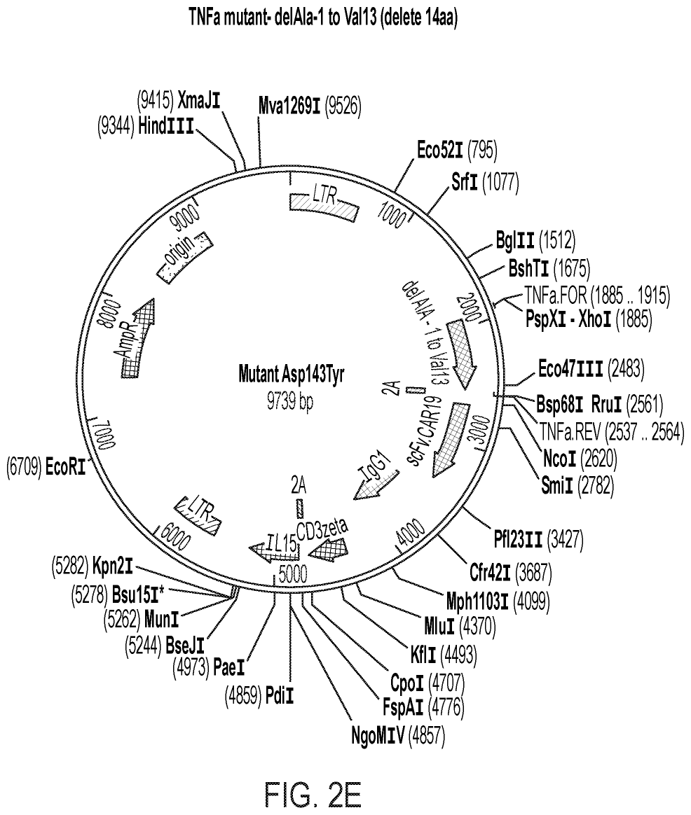

FIGS. 2A, 2B, 2C, 2D, and 2E provide examples of vectors that may encode the TNF-alpha mutants. FIG. 2A illustrates a vector map example of a TNF-alpha mutant having deletions of amino acids Val1 and Pro12, and the mutant is co-expressed with a CD19-specific CAR and is also co-expressed with IL-15, all as separate polypeptides, as an example. FIG. 2B illustrates a vector map example of a TNF-alpha mutant having a deletion at Valine 13, and the mutant is separately co-expressed with a CD19-specific CAR and separately co-expressed with IL-15, as an example. FIG. 2C illustrates a vector map example of a TNF-alpha mutant having deletions of amino acids Val1 and Val 13, and the mutant is separately co-expressed with a CD19-specific CAR and IL-15, as an example. FIG. 2D illustrates a vector map example of a TNF-alpha mutant having deletions of amino acids Val1 through to Val 13 (13 aa deletions), and the mutant is separately co-expressed with a CD19-specific CAR and IL-15, as an example. FIG. 2E illustrates a vector map example of a TNF-alpha mutant having deletions of amino acids Ala-1 through to Val 13 (14 aa deletion), and the mutant is separately co-expressed with a CD19-specific CAR and IL-15, as an example.

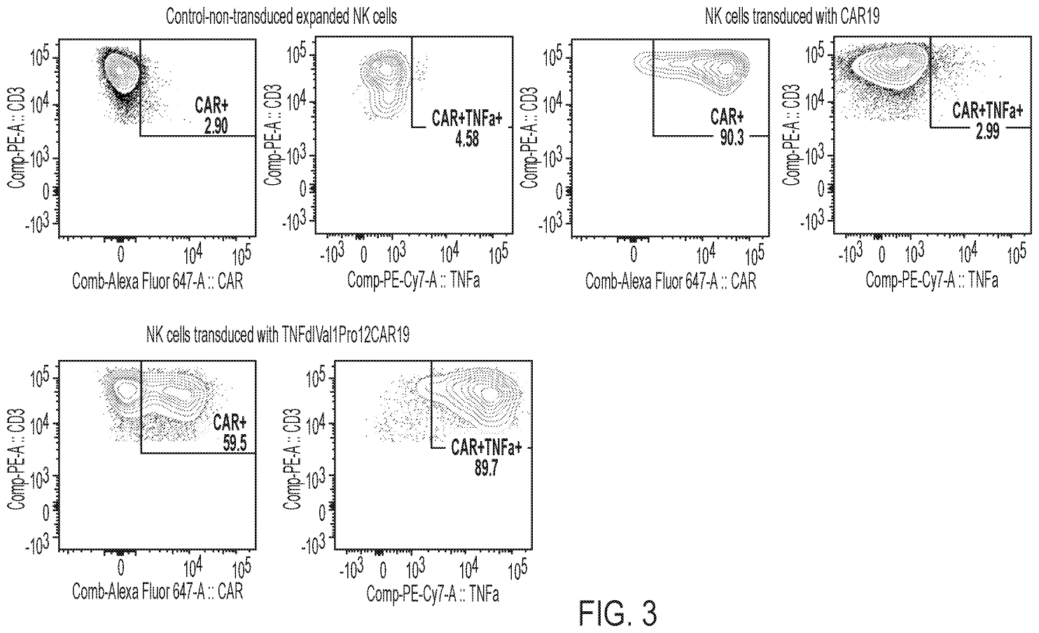

The mutated uncleavable TNF-alpha (in cells transduced with a vector encoding both TNF-alpha mutant with deletions at Val1 and Pro12 and a CD19-specific CAR, as an example) is stably expressed on the cell surface after, for example, viral transduction or electroporation of its encoding sequence (FIG. 3).

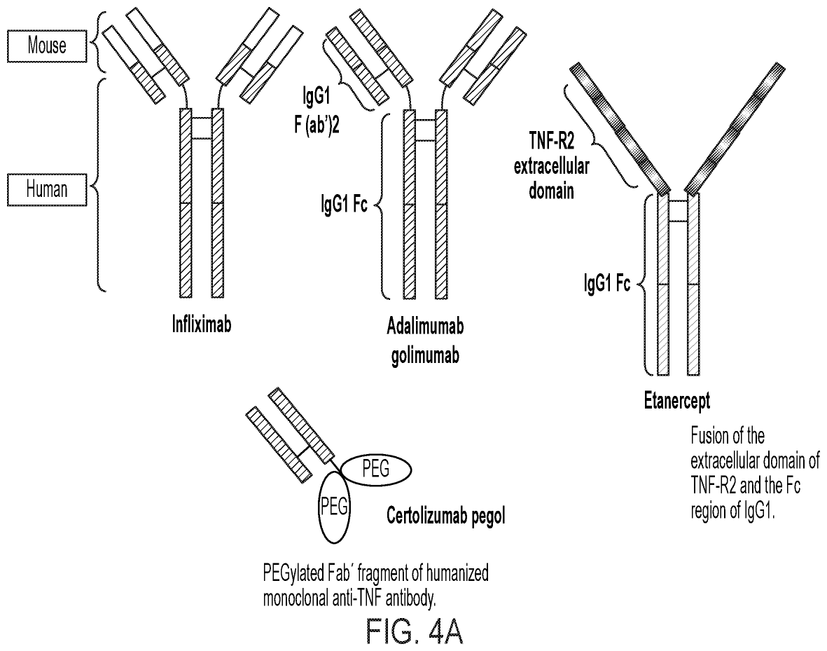

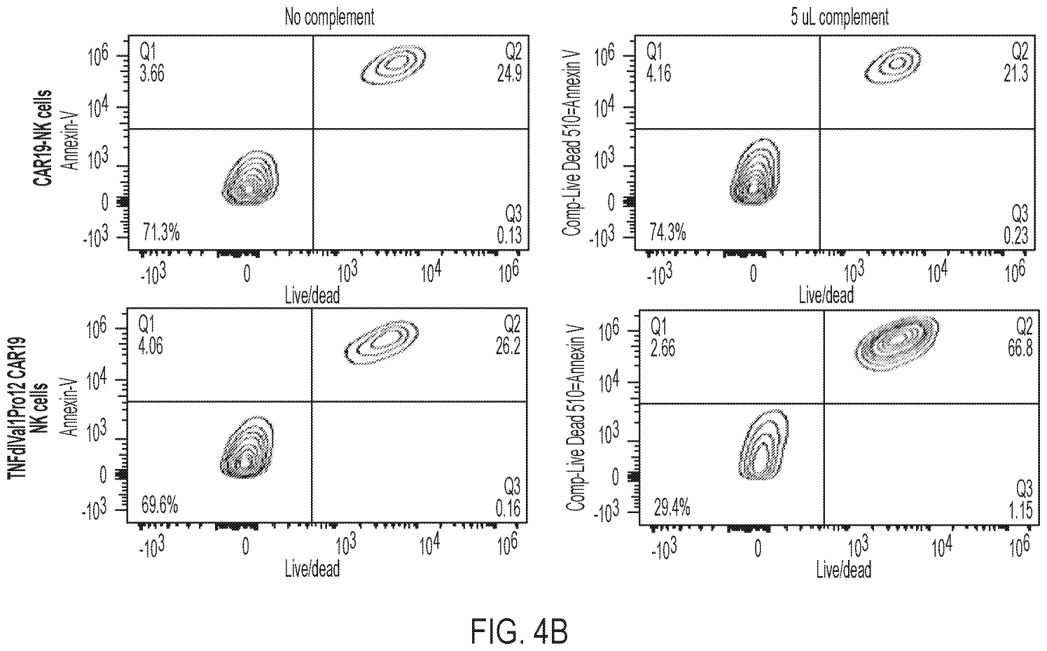

Cells expressing the uncleavable TNF-α mutants can be targeted for selective deletion using FDA-approved TNF-α antibodies (for example), such as etanercept, infliximab or adalilumab. FIG. 4A illustrates examples of anti-TNF antibodies. FIG. 4B demonstrates that greater than 70% of NK cells expressing mutant TNF-alpha are eliminated by complement dependent cytotoxicity (CDC) within 90 minutes of treatment with infliximab.

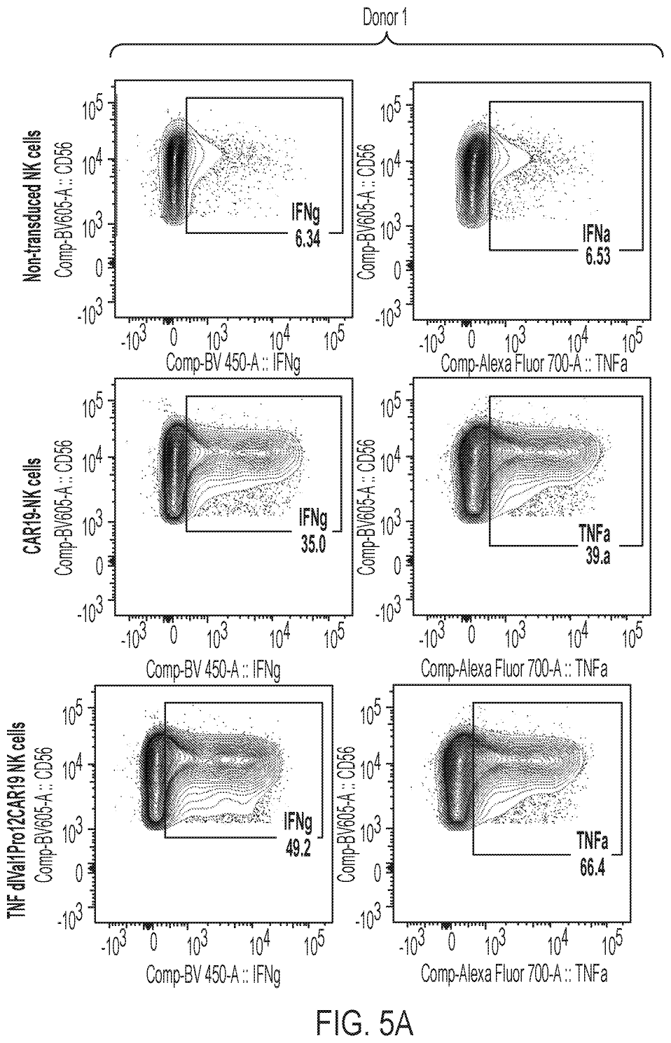

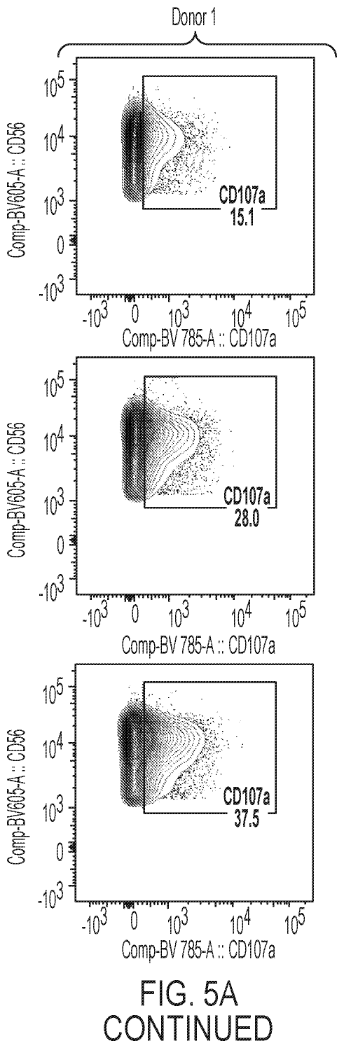

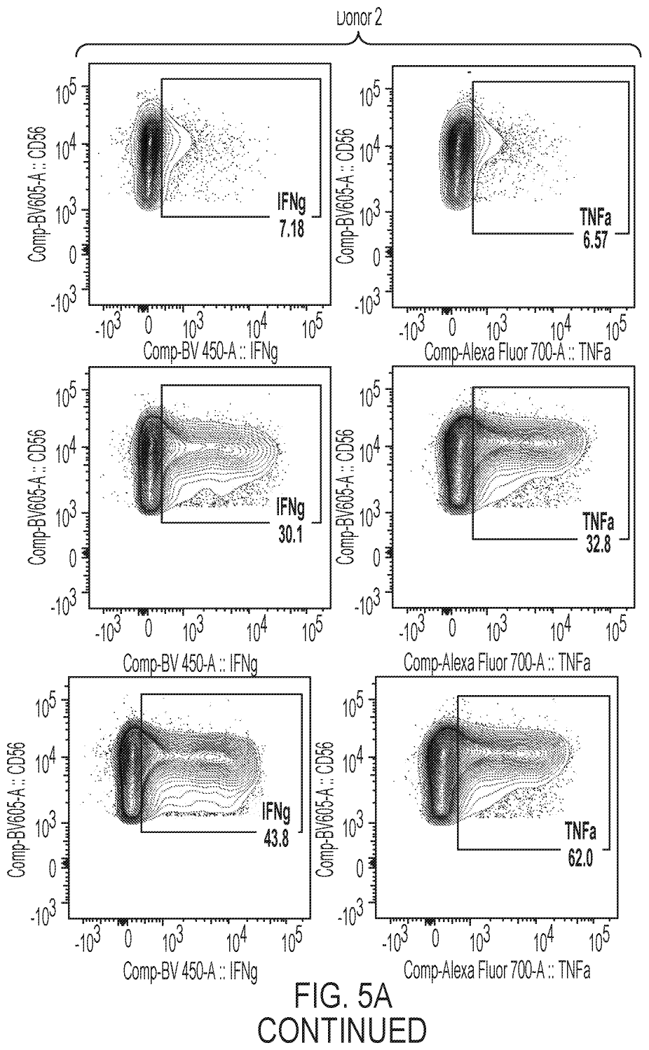

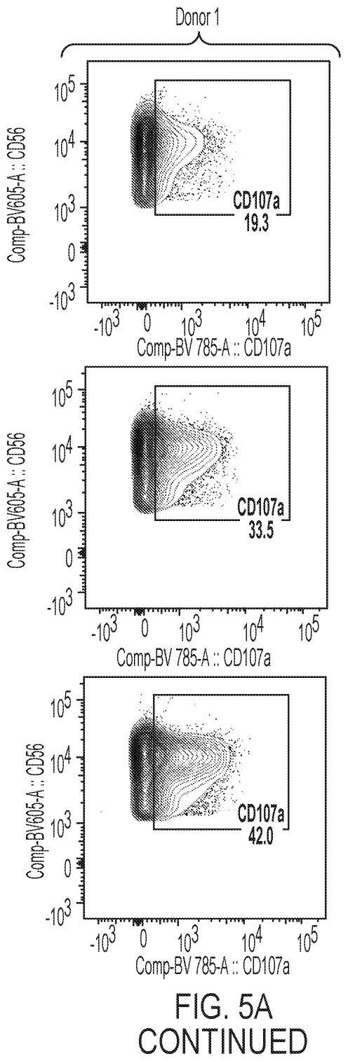

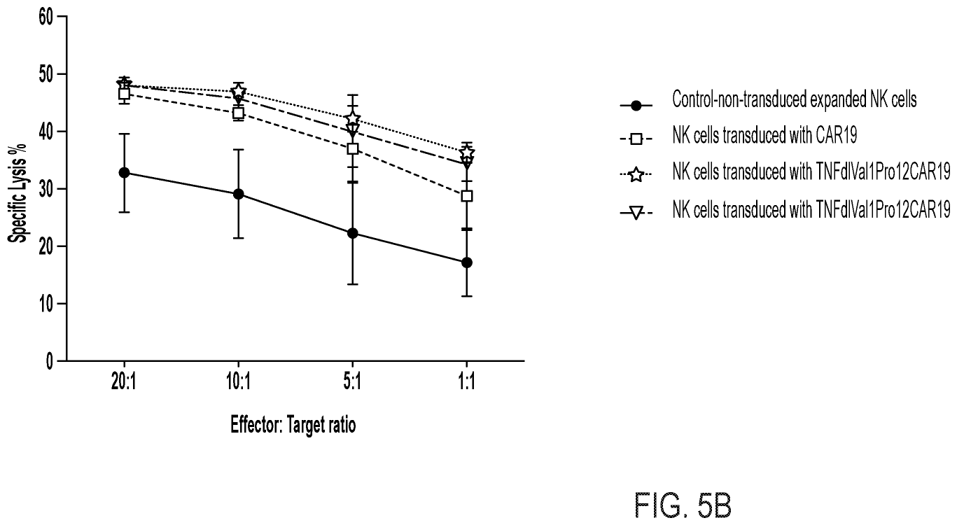

FIG. 5A demonstrates that in response to Raji targets, the NK cells transduced with a vector that co-expresses TNF-alpha mutant and an CD19-specific CAR produce more effector cytokines and degranulate more efficiently when compared to NK cells that express the anti-CD19 CAR alone. In FIG. 5B, Raji targets are efficiently killed by NK cells transduced with a vector that separately co-expresses a TNF-alpha mutant (deletion of Val1 and Pro12, as an example) and a CD19-specific CAR. The TNF-alpha mutant protein with deletions of Valine at position 1 Proline at position 12 is biologically active and mediates a strong anti-tumor response upon direct cell-cell contact, further contributing to the antitumor activity of the transduced cells.

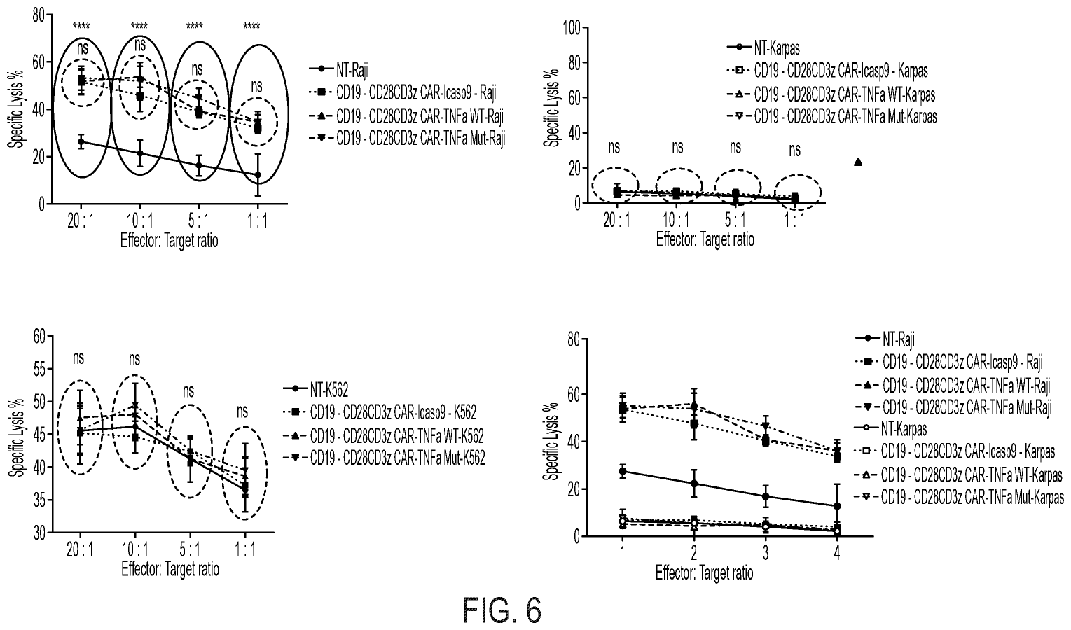

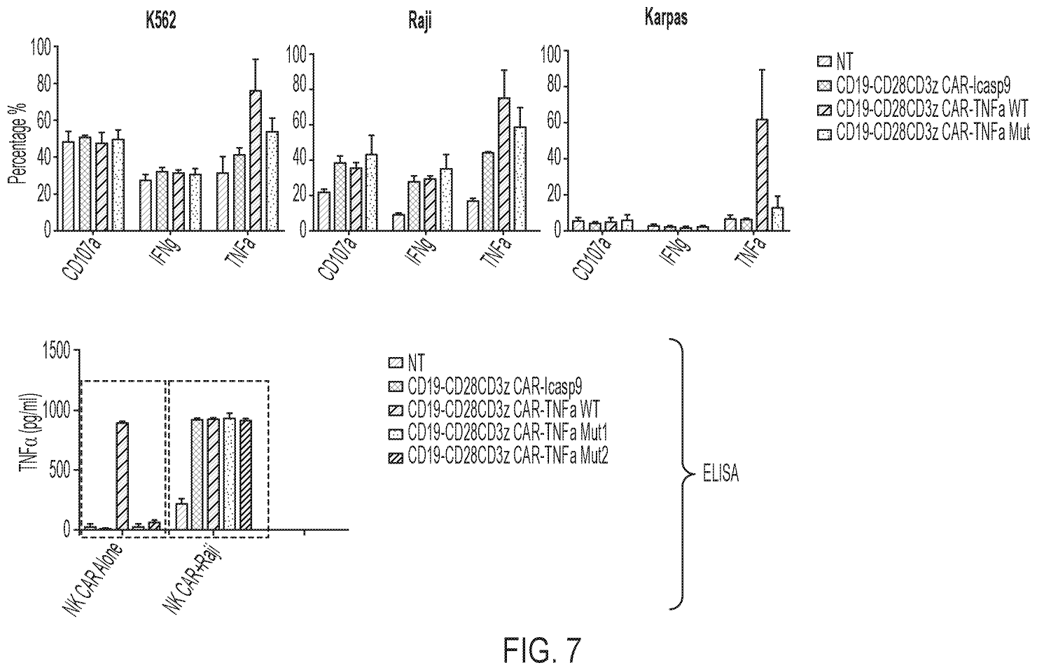

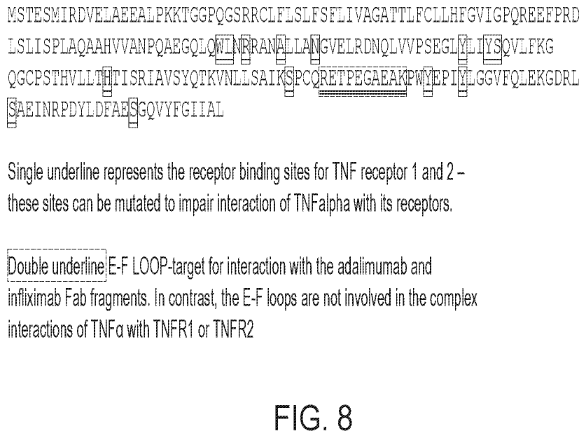

The transduced NK cells harboring a vector that separately expresses a CD19-specific CAR and a TNF-alpha mutant do not exhibit off-target activity (FIG. 6). FIG. 7 demonstrates that NK cells transduced with a vector that separately expresses a CD19-specific CAR and a TNF-alpha mutant do not exhibit off-target activity and do not secrete TNF-alpha non-specifically. FIG. 8 illustrates that TNF-alpha receptor binding sites for TNF receptors 1 and 2 vs. TNF-alpha antibodies infliximab and adalimumab are different. This demonstrates that the mutations in the TNFalpha gene will not negatively impact the ability of TNFalpha antibodies in recognizing the TNFa mutant protein; i.e. the TNFalpha mutant can still be used as a suicide gene and be targeted by the antibodies.

Additional safety studies may be employed. For example, in vivo murine toxicity studies with CD19-specific CAR NK cells may be performed. For example, in an established Raji NSG mouse model one can compare TNF-alpha WT vs. TNF-alpha mutant, CD19-specific CAR NK cells also expressing IL15. However, these mutants were previously tested in mice and their safety was demonstrated (Karp et al., 1992).

One may employ synapse and signaling studies to characterize interaction of TNF-alpha mutant vs. TNF-alpha wild type vs. exogenous TNF-alpha with TNF-alpha receptor 1 (TNF-R1) and TNF-alpha receptor 2 (TNF-R2). Such studies may incorporate measurement of apoptosis induction and caspase (downstream of TNF-R1) in Ramos cells (which express TNF R1 but not TNFR2). In addition or alternatively, one can measure NFkappaB in Jurkat cells that express both TNFR2 and TNFR1.

### Example 2

### Comparison of Antitumor Activity of CAR-NK Cells Transduced with TNFAmut-CAR19-IL15 Vs. IC9-CAR19-IL15 Construct

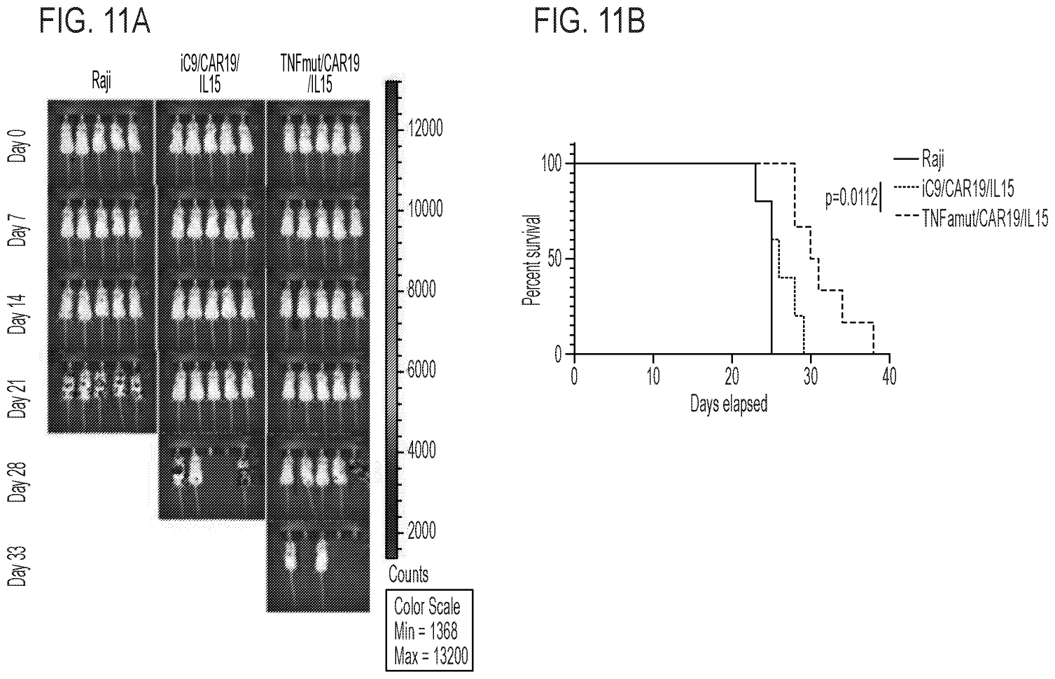

FIG. 11 provides a comparison of antitumor activity of CAR-NK cells from cord blood transduced with either TNF-alpha mut-CAR19-IL15 construct or inducible caspase 9 (iC9)-CAR19-IL15 constructs. In FIG. 11A, NSG mice with Raji tumor received 3×10e6 CAR cord blood NK cells transduced with TNF-alpha mut-CAR19-IL15 construct or transduced with iC9-CAR19-IL15 construct. FIG. 11B demonstrates percent survival over time. Mice transduced with TNF-alpha mut-CAR19-IL15 construct outlived control mice and mice transduced with iC9-CAR19-IL15 construct.

### REFERENCES

All patents and publications mentioned in the specification are indicative of the level of those skilled in the art to which the embodiments of the disclosure pertain. All patents and publications are herein incorporated by reference in their entirety to the same extent as if each individual publication was specifically and individually indicated to be incorporated by reference.

### PATENTS

U.S. Pat. No. 5,118,500
U.S. Pat. No. 6,143,866

### PUBLICATIONS

Karp, Stephen E., Hwu, Patrick, et al. (1992) In vivo Activity of Tumor Necrosis Factor (TNF) Mutants: Secretory but non Membrane-Bound TNF Mediates the Regression of Retrovirally Tranduced Murine Tumor. J. Immunol., vol. 149(6): 2076-2081.
Perez, C., Albert, I. et al. (1990) A Nonsecretable Cell Surface Mutant of Tumor Necrosis Factor (TNF) Kills by Cell-to-Cell Contact. Cell, vol. 63, 251-258.

Although the present disclosure and its advantages have been described in detail, it should be understood that various changes, substitutions and alterations can be made herein without departing from the spirit and scope of the design as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the present disclosure, processes, machines, manufacture, compositions of matter, means, methods, or steps, presently existing or later to be developed that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A composition comprising a transduced cell comprising a nucleic acid that encodes one or more engineered nonsecretable tumor necrosis factor (TNF)-alpha mutant polypeptides and a nucleic acid that encodes one or more therapeutic gene products.

2. The composition of claim 1, wherein the TNF-alpha mutant polypeptide comprises a deletion with respect to SEQ ID NO:8 of the following:
amino acid residue 1 and amino acid residue 12;
amino acid residue 1 and amino acid residue 13;
amino acid residues 1-12;
amino acid residues 1-13; or
amino acid residues −1 to 13.

3. The composition of claim 1, wherein the therapeutic gene product is an engineered receptor.

4. The composition of claim 1, wherein the engineered receptor is a T-cell receptor, chimeric antigen receptor (CAR), cytokine receptor, homing receptor, or chemokine receptor.

5. The composition of claim 3, wherein the engineered receptor targets a cancer antigen.

6. The composition of claim 3, wherein the engineered receptor is a CAR that comprises one or more costimulatory domains.

7. The composition of claim 6, wherein the one or more costimulatory domains comprises the costimulatory domain of CD28, DAP12, CD137 (4-1BB), CD134 (OX40), Dap10, CD27, CD2, CD5, ICAM-1, LFA-1 (CD11a/CD18), Lck, TNFR-I, TNFR-II, Fas, CD30, CD40 or a combination thereof.

8. The composition of claim 1, wherein the nucleic acid that encodes the TNF-alpha mutant polypeptide and the nucleic acid that encodes the therapeutic gene product are the same nucleic acid molecule.

9. The composition of claim 1, wherein the nucleic acid that encodes the TNF-alpha mutant polypeptide and the nucleic acid that encodes the therapeutic gene product are different nucleic acid molecules.

10. The composition of claim 8, wherein the nucleic acid molecule is a vector.

11. The composition of claim 10, wherein the vector is a viral vector or a non-viral vector.

12. The composition of claim 11, wherein the viral vector is a retroviral vector, lentiviral vector, adenoviral vector, or adeno-associated viral vector.

13. The composition of claim 11, wherein the non-viral vector is a plasmid, lipid, or transposon.

14. The composition of claim 1, wherein the cell is an immune cell or a stem cell.

15. The composition of claim 14, wherein the immune cell is a T cell, a NK cell, NKT cell, iNKT cell, B cell, regulatory T cell, monocyte, macrophage, dendritic cell, or mesenchymal stromal cell.

16. The composition of claim 1, wherein the TNF-alpha mutant polypeptide comprises SEQ ID NO:1, SEQ ID NO:3, SEQ ID NO:5, SEQ ID NO:39 or SEQ ID NO:41.

17. The composition of claim 1, wherein the TNF-alpha mutant polypeptide is encoded by a sequence that comprises SEQ ID NO:2, SEQ ID NO:4, SEQ ID NO:6, SEQ ID NO:38, or SEQ ID NO:40.

18. The composition of claim 1, wherein the cell expresses an exogenously provided cytokine.

19. The composition of claim 18, wherein the cytokine is IL-7, IL-2, IL-15, IL-12, IL-18, IL-21 or a combination thereof.

20. The composition of claim 18, wherein the cytokine is encoded from the same vector as the TNF-alpha mutant gene.

21. The composition of claim 18, wherein the cytokine is expressed as a separate polypeptide molecule as the TNF-alpha mutant and as a separate polypeptide molecule as an engineered receptor of the cell.

22. The composition of claim 1, wherein the TNF-alpha mutant polypeptide lacks one or more further mutations that prevent binding of the TNF-alpha mutant polypeptide to a TNF receptor.

23. A method of inducing death for a transduced cell expressing an engineered nonsecretable TNF-alpha mutant polypeptide, comprising the step of providing an effective amount of at least one agent that binds the TNF-alpha mutant on the transduced cell.

24. The method of claim 23, wherein the agent that binds TNF-alpha is an antibody, small molecule, polypeptide, nucleic acid, or combination thereof.

25. The method of claim 24, wherein the antibody is a monoclonal antibody.

26. The method of claim 23, wherein the cell further expresses an engineered receptor.

27. The method of claim 26, wherein the engineered receptor is a T-cell receptor or a CAR.

28. The method of claim 26, wherein the engineered receptor targets a cancer antigen.

29. The method of claim 23, wherein the method occurs in vivo in an individual with a medical condition and the individual has been provided a therapy for the medical condition that comprises a plurality of the transduced cells.

30. The method of claim 29, wherein the medical condition is cancer.

31. The method of claim 29, wherein the agent is provided to the individual upon onset of one or more adverse events from the therapy.

32. The method of claim 31, wherein the individual exhibits one or more symptoms of cytokine release syndrome, neurotoxicity, anaphylaxis/allergy, and/or on-target/off tumor toxicity.

33. The method of claim 29, wherein the individual has been provided, is provided, and/or will be provided an additional therapy for the medical condition.

34. The method of claim 23, wherein the TNF-alpha mutant polypeptide lacks one or more further mutations that prevent binding of the TNF-alpha mutant polypeptide to a TNF receptor or prevents reverse signaling.

35. A method of reducing the effects of cytokine release syndrome in an individual that has received and/or who is receiving cell therapy with cells that express a nonsecretable TNF-alpha mutant, comprising the step of providing an effective amount of one or more agents that bind the mutant to cause in the individual (a) elimination of at least some of the cells of the cell therapy; and (b) reduction in the level of soluble TNF-alpha.

36. A method of reducing the risk of toxicity of a cell therapy for an individual, comprising the step of modifying the cells of the cell therapy to express a nonsecretable TNF-alpha mutant.

37. The method of claim 36, wherein the cell therapy is for cancer.

38. The method of claim 36, wherein the cell therapy comprises an engineered receptor that targets an antigen.

39. A vector, comprising a sequence that encodes a nonsecretable TNF-alpha mutant and that encodes an engineered receptor.

40. The vector of claim 39, wherein the nonsecretable TNF-alpha mutant and the engineered receptor are encoded from the vector as separate polypeptides.

41. The vector of claim 39, wherein sequence of the vector that encodes the nonsecretable TNF-alpha mutant and sequence of the vector that encodes the engineered receptor are separated on the vector by a 2A element or an IRES element.

42. The vector of claim 39, wherein the engineered receptor is a CAR.

43. The vector of claim 39, wherein the vector further encodes a cytokine.

44. The vector of claim 43, wherein the cytokine is IL-7, IL-2, IL-15, IL-12, IL-18, or IL-21.

45. The vector of claim 43, wherein the cytokine is expressed from the vector as a separate polypeptide as the TNF-alpha mutant and the engineered receptor.

46. As a composition of matter, a nucleic acid sequence comprising SEQ ID NO:15.

47. As a composition of matter, a nucleic acid sequence comprising SEQ ID NO:16.
